(19) United States
(12) Patent Application Publication
(10) Pub. No.: US 2026/0085108 A1
(43) Pub. Date: Mar. 26, 2026

(54) METHODS OF SEPARATING CHELATOR

(71) Applicant: Bristol-Myers Squibb Company, Princeton, NJ (US)

(72) Inventors: Jason HUANG, Princeton, NJ (US); Qiang LI, Princeton, NJ (US)

(21) Appl. No.: 19/109,768

(22) PCT Filed: Sep. 8, 2023
(86) PCT No.: PCT/US2023/073772

§ 371 Date: Mar. 7, 2025

Related U.S. Application Data
(60) Provisional application No. 63/375,179, filed on Sep. 9, 2022.

(51) Int. Cl. C07K 16/00; B01D 15/14; B01D 15/30; B01D 15/42; C07K 1/22; G01N 30/02; G01N 30/16; G01N 30/52; G01N 30/74

(52) U.S. Cl. CPC C07K 16/00; B01D 15/14; B01D 15/305; B01D 15/424; C07K 1/22; G01N 30/16; G01N 30/52; G01N 30/74; G01N 2030/027; G01N 2030/525

(57) ABSTRACT

The present disclosure is directed to methods of separating a chelator from a mixture and measuring the concentration of the chelator in the mixture using a chromatography column. Such methods can be useful in adjusting the concentration of a chelator of a composition comprising a protein.

### CROSS-REFERENCE TO RELATED APPLICATIONS

This application claims the benefit of U.S. Provisional Application No. 63/375,179, filed Sep. 9, 2022, which is incorporated herein by reference in its entirety.

### FIELD OF THE DISCLOSURE

The present application relates to the field of analyzing the content of a chelator in a mixture containing proteins using chromatography columns.

### BACKGROUND OF THE DISCLOSURE

Chelators are commonly used in biologics formulations to prevent oxidation induced by metal ions and therefore improve the stability of biologics in the formulation. HPLC and UV-vis spectrometry were reported for simultaneous determination of diethylene triamine pentaacetic acid (DTPA) and other polyaminocarboxylate (PAC) chelators. Other techniques for the analysis of chelators include voltammetry, potentiometry, gas chromatography/mass spectrometry, ion chromatography, and capillary electrophoresis. In HPLC, chelating agents such as DTPA, ethylenediamine tetraacetic acid (EDTA), and nitrilotriacetic acid (NTA) were often analyzed as Fe(III) or Cu(II) complexes in reversed-phase or ion pairing mode with UV detection. Traditional chromatography methods typically require multiple steps, including a dilution step and an elution step. Recently, ultra performance liquid chromatography (UPLC) is used to separate a chelator and a protein in a sample by exploiting the size exclusion effect of a narrow pore RP-HPLC column to help elute the large mAb at the void volume. This method enables direct injections of protein samples for chelators analysis in biologics formulations. However, the result is not satisfying, as the analytical column needs to be replaced after about 30 injections to meet the performance requirements in high protein concentration formulations.

As such, there remains a need in the field of biologics formulations for a rapid and reliable method for monitoring chelator concentration during formulation and process development and in the control of the chelator levels in the final biologics drug products.

### BRIEF SUMMARY OF THE DISCLOSURE

The present disclosure provides a method of separating a chelator in a mixture comprising a protein and the chelator, the method comprising contacting the mixture with a chromatography column, wherein the chromatography column comprises a stationary phase, wherein the stationary phase:
has a charge, which is the same as the charge of the protein,
is capable of forming a non-polar interaction with the chelator, and
comprises a porous surface with a pore size that is less than the diameter of the protein.

In some aspects, the method further comprises contacting the chromatography column with an elution buffer, wherein the contacting with the elution buffer occurs after the contacting of the chromatography column with the mixture.

In some aspects, after contacting the chromatography column with the elution buffer, the protein is selectively eluted from the chromatography column while the chelator is retained within the chromatography column.

In some aspects, the method further comprises eluting the chelator from the chromatography column.

In some aspects, the method further comprises measuring the concentration of the chelator in the mixture

In some aspects, measuring the concentration of the chelator in the mixture comprises contacting a standard solution with the chromatography column, wherein the standard solution comprises the chelator.

In some aspects, the standard is retained within the chromatography column for at least about 3.5 min.

In some aspects, the standard is retained in the column for about 3.5 min, for about 3.6 min, for about 3.7 min, for about 3.8 min, for about 3.9 min, for about 4.0 min, or for about 4.1 min.

In some aspects, the contacting of the mixture with the chromatography column comprises directly injecting the mixture into the chromatography column.

In some aspects, prior to the contacting of the mixture with the chromatography column, the mixture is diluted in a dilution buffer.

In some aspects, the mixture is diluted in the dilution buffer at about 1:1 dilution, at about 1:2 dilution, at about 1:3 dilution, at about 1:4 dilution, at about 1:5 dilution, at about 1:6 dilution, at about 1:7 dilution, at about 1:8 dilution, at about 1:9 dilution, at about 1:10 dilution, or at about 1:20 dilution, at about 1:30 dilution, at about 1:40 dilution, at about 1:50 dilution, at about 1:60 dilution, at about 1:70 dilution, at about 1:80 dilution, at about 1:90 dilution, at about 1:100 dilution, at about 1:200 dilution, at about 1:300 dilution, at about 1:400 dilution, or at about 1:500 dilution.

In some aspects, the method further comprises (i) obtaining a peak response from the mixture (rᵤ), (ii) obtaining a peak response from the standard solution (rₛ), (iii) or both (i) and (ii), wherein the peak response is the peak area as determined by high performance liquid chromatography (“HPLC”) or ultra performance liquid chromatography (“UPLC”) at the wavelength of 260 nm.

In some aspects, the rᵤ and the rₛ are obtained by HPLC or UPLC.

In some aspects, the concentration (μg/mL) of the chelator is calculated as formula (I):

$$C ( {V}_{u} / {V}_{n} ) ( {r}_{u} / {r}_{s} )$$

wherein C is the concentration (μg/mL) of the chelator in the standard solution; rᵤ and rₛ are the peak responses obtained from the mixture and the standard solution, respectively, Vᵤ is the total volume (μL) of the mixture after being diluted; Vₙ is the volume (μL) of the mixture before the dilution,
wherein C is calculated as formula (II):

$$[ {W}_{s} \times P ] \times D \times 1000 / {V}_{s}$$

wherein Ws is the weight (mg) of a reference material for the chelator, P is the purity of the reference material (expressed as a fraction), Vs is the volume of the standard solution (mL). D is the Dilution Factor (1/100).

In some aspects, the present disclosure provides a method of adjusting the concentration of a chelator in a composition comprising a protein, the method comprising measuring the concentration of the chelator present in the composition, wherein the concentration of the chelator present in the composition is measured according to any of the methods above.

In some aspects, the present disclosure also provides a method of producing a composition comprising a protein, the method comprising measuring a concentration of a chelator present in the composition, wherein the concentration of the chelator present in the composition is measured according to any of the methods above.

In some aspects, the method comprises increasing the concentration of the chelator if the measured concentration of the chelator is less than a reference amount (e.g., the concentration of the chelator present in a standard solution).

In some aspects, the measured concentration of the chelator is less than about 5%, less than about 10%, less than about 20%, less than about 30%, less than about 40%, less than about 50%, less than about 60%, less than about 70%, less than about 80%, less than about 90%, or about 100% as compared to the reference amount.

In some aspects, increasing the concentration of the chelator comprises adding an amount of the chelator to the composition.

In some aspects, the method further comprises an additional measuring of the concentration of the chelator present in the composition after adding the amount of the chelator to the composition.

In some aspects, the additional measuring is performed according to any of the methods above.

In some aspects, after adding the amount of the chelator to the composition, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference.

In some aspects, the method further comprises decreasing the concentration of the chelator if the measured concentration of the chelator is higher than the reference amount.

In some aspects, the measured concentration of the chelator is greater than about 5%, greater than about 10%, greater than about 20%, greater than about 30%, greater than about 40%, greater than about 50%, greater than about 60%, greater than about 70%, greater than about 80%, greater than about 90%, greater than about 100%, greater than about 125%, greater than about 150%, greater than about 175%, or greater than about 200% as compared to the reference amount.

In some aspects, decreasing the concentration of the chelator comprises adding a diluent to the composition, increasing the concentration of the protein in the composition, or both.

In some aspects, the method further comprises an additional measuring of the concentration of the chelator present in the composition after the decreasing.

In some aspects, the additional measuring is performed according to the method of any one of claims 1 to 14.

In some aspects, after the decreasing, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference.

In some aspects, the chromatography column and the protein have positive charge.

In some aspects, the chromatography column is capable of eluting a protein, wherein the diameter of the protein is less than about 70 Å, less than about 80 Å, less than about 90 Å, less than about 100 Å, less than about 110 Å, less than about 120 Å, less than about 130 Å, less than about 140 Å, less than about 150 Å, less than about 160 Å, less than about 170 Å, or less than about 180 Å.

In some aspects, the chromatography column is capable of eluting a protein, wherein the diameter of the protein is between about 70 Å and about 180 Å, about between 70 Å and about 170 Å, between about 70 Å and about 160 Å, between about 70 Å and about 150 Å, between 70 about Å and about 140 Å, between about 70 Å and about 130 Å, between about 70 Å and about 120 Å, between about 70 Å and about 110 Å, between about 70 Å and about 100 Å, between about 80 Å and about 180 Å, between about 80 Å and about 170 Å, between about 80 Å and about 160 Å, between about 80 Å and about 150 Å, between about 80 Å and about 140 Å, between about 80 Å and about 130 Å, between about 80 Å and about 120 Å, between about 80 Å and about 110 Å, between about 80 Å and about 100 Å, between about 90 Å and about 180 Å, between about 90 Å and about 170 Å, between about 90 Å and about 160 Å, between about 90 Å and about 150 Å, between about 90 Å and about 140 Å, between about 90 Å and about 130 Å, between about 90 Å and about 120 Å, between about 90 Å and about 110 Å, between about 90 Å and about 100 Å, between about 100 Å and about 180 Å, between about 100 Å and about 170 Å, between about 100 Å and about 160 Å, between about 100 Å and about 150 Å, between about 100 Å and about 140 Å, between about 100 Å and about 130 Å, between about 100 Å and about 120 Å, between about 100 Å and about 110 Å, between about 110 Å and about 180 Å, between about 110 Å and about 170 Å, between about 110 Å and about 160 Å, between about 110 Å and about 150 Å, between about 110 Å and about 140 Å, between about 110 Å and about 130 Å, between about 110 Å and about 120 Å, between about 120 Å and about 180 Å, between about 120 Å and about 170 Å, between about 120 Å and about 160 Å, between about 120 Å and about 150 Å, between about 120 Å and about 140 Å, between about 120 Å and about 130 Å, between about 130 Å and about 180 Å, between about 130 Å and about 170 Å, between about 130 Å and about 160 Å, between about 130 Å and about 150 Å, between about 130 Å and about 140 Å, between about 140 Å and about 180 Å, between about 140 Å and about 170 Å, between about 140 Å and about 160 Å, between about 140 Å and about 150 Å, between about 150 Å and about 180 Å, between about 150 Å and about 170 Å, between about 150 Å and about 160 Å, between about 160 Å and about 180 Å, between about 160 Å and about 170 Å, or between about 170 Å and about 180 Å.

In some aspects, the pore size is no more than about 110 Å.

In some aspects, the pore size is about 110 Å, about 100 Å, about 90 Å, about 80 Å, or about 70 Å.

In some aspects, the chelator comprises DTPA, EDTA, or both.

In some aspects, the chromatography column is an hydrophilic interaction chromatography (HILIC) column.

In some aspects, the chromatography column is packed with a stationary phase comprising multimode hydrophobic ligand and a positive charged terminal functional group.

In some aspects, the positive charged terminal functional group comprises an amino group.

In some aspects, the column is Newcrom BH column.

In some aspects, the chromatography is performed using a mobile phase comprising a buffer.

In some aspects, the buffer comprises acetonitrile, sulfuric acid, FeCl₃, or a combination thereof.

In some aspects, the acetonitrile is present in the buffer at a concentration of about 0.5%, about 1%, about 1.5%, about 2%, about 2.5%, or about 3%.

In some aspects, the sulfuric acid is present in the buffer at a concentration of about 0.05%, about 0.1%, about 0.2%, about 0.3%, about 0.4%, about 0.5%, about 0.6%, about 0.7%, about 0.8%, about 0.9%, or about 1%.

In some aspects, the sulfuric acid is present in the buffer at a concentration of about 0.15% to 0.20%.

In some aspects, the FeCl₃ is present in the buffer at a concentration of about 0.1 mM, about 0.2 mM, about 0.3 mM, about 0.4 mM, about 0.5 mM, about 0.6 mM, about 0.7 mM, about 0.8 mM, about 0.9 mM, or about 1.0 mM.

In some aspects, the buffer comprises 2% Acetonitrile, 0.2% sulfuric acid, and 0.6 mM FeCl₃.

In some aspects, the buffer comprises 2% Acetonitrile, 0.15% sulfuric acid, 0.6 mM FeCl₃.

In some aspects, the buffer has a pH from about 1.0 to about 5.0, or 1.0 to about 4.5, or about 1.0 to about 4.0.

In some aspects, the buffer has a pH of about 0.5, about 1.0, about 1.5, about 2.0, about 2.5, about 3.0, about 3.5, about 4.0, about 4.5, or about 5.0.

In some aspects, the buffer has a pH of about 1.0 to about 4.0, or any pH in between.

In some aspects, the dilution buffer is the buffer of any one of claims 40-47.

In some aspects, the concentration of the protein in the mixture is about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, or about 300 mg/mL.

In some aspects, the protein is conjugated to a drug, such that the mixture or the composition comprises a protein-drug conjugate (PDC).

In some aspects, the protein-drug conjugate comprises an antibody-drug conjugate (ADC).

In some aspects, the protein comprises an antibody or antigen binding portion thereof.

In some aspects, the protein comprises a fusion protein.

In some aspects, the fusion protein comprises a half-life extending moiety.

In some aspects, the half-life extending moiety comprises an Fc.

In some aspects, the fusion protein comprises an Fc fused to an antibody or antigen binding portion thereof.

In some aspects, present disclosure provides a composition produced according to the methods described herein.

In some aspects, the composition further comprises a pharmaceutically acceptable excipient.

### BRIEF DESCRIPTION OF THE DRAWINGS/FIGURES

FIG. 1 provides a scheme showing the characteristics of exemplary packing material of chromatography columns that are useful for the methods provided herein. As illustrated, in some aspects, the chromatography column has a positive charge (represented by the +symbols) and a selective pore size (e.g., 100 Å), such that a protein can be eluted based on both charge repulsion and size-exclusion.

FIG. 2 shows chromatograms displaying the elution profiles of a blank (“Blank”; 3^{rd} line from the top), a DTPA standard (“DTPA STD”; 2^{nd} line from the top), and two different batches (B1 and B2) of antibody formulations (i.e., (i) “mAb-1 B2”; bottom line; and (ii) “mAb-1 B1”; top line) observed on a SIELC Newcrom BH column (150 mm×3.2 mm i.d., 3 μm particle size, 100 Å pore size) described herein at the wavelength of 260 nm.

FIGS. 3A and 3B show the repeatability and robustness of the HILIC chromatography columns described herein. In particular, FIG. 3A provides a chromatogram of the elution profile of a DTPA standard added to the column after the 88th injection and elution of a mAb-1 formulation sample. FIG. 3B provides a chromatogram of the elution profile observed after the 88th injection of the mAb-1 formulation sample to the HILIC chromatography column.

FIGS. 3C and 3D show the challenge for DTPA analysis in high concentration mAb-1 formulations by UPLC method using a narrow pore C18 column. FIG. 3C provides overlay of chromatograms of the elution profiles observed after the first (light blue), 10th (red), and 30th (black) injections of mAb-1 samples. FIG. 3D provides overlay of chromatograms of the elution profiles observed after the first (black) and 31 st injections (dark blue) of DTPA standard injections.

FIGS. 4A and 4B provide the chromatograms showing the elution profile of a 4 μg/mL DTPA standard solution and a 5× dilution of a mAb-4 formulation solution comprising 16 mg/mL mAb-4 and 8 μg/mL DTPA, respectively.

FIGS. 5A and 5B provide linearity of curve of DTPA obtained in the range of 25%-200% of working concentration (4 μg/mL) with HILIC HPLC chromatography. FIG. 5A is a linear plot of the peak area against the increasing concentration of DTPA. FIG. 5B is the overlay of the elution profile of DTPA at different concentrations.

FIGS. 6A and 6B provide evaluation of the precision of the method with HILIC HPLC chromatography. FIG. 6A shows the overlapping elution profiles of 6 repeated standard injections. The relative standard deviation (RSD) is 0.1%. FIG. 6B shows the overlapping elution profiles of 3 repeated sample injections. The relative standard deviation (RSD) is 0.7%.

FIG. 7 provides chromatograms showing the elution profiles of sample containing monoclonal antibody (mAb) and ethylenediamine tetraacetic acid (EDTA) (top line) or EDTA standard alone (bottom line) observed on a SIELC Newcrom BH column (150 mm×3.2 mm, 3 μm particle size, 100 Å) described herein at the wavelength of 260 nm.

FIG. 8 shows chromatograms showing the elution profiles of EDTA under DTPA analytical conditions. The top line is DTPA (Retention time at 3.8) and the bottom one is EDTA (Retention time at 3.2). EDTA concentration is 20 μM (5.8 μg/mL) and DTPA is 4.0 μg/mL.

### DETAILED DESCRIPTION OF THE DISCLOSURE

The present disclosure is generally directed to methods of measuring the concentration of chelators in biologics formulations. More particularly, the methods provided herein comprise separating a chelator and a protein in a mixture with a chromatography column which (1) has the same charge of the protein, (2) is capable of forming a non-polar interaction with the chelator, and (3) comprises a porous surface with a pore size that is less than the diameter of the protein. As demonstrated herein, in some aspects, by separating the chelator and protein in a mixture, it is possible to efficiently measure the concentration of the chelator present in the mixture.

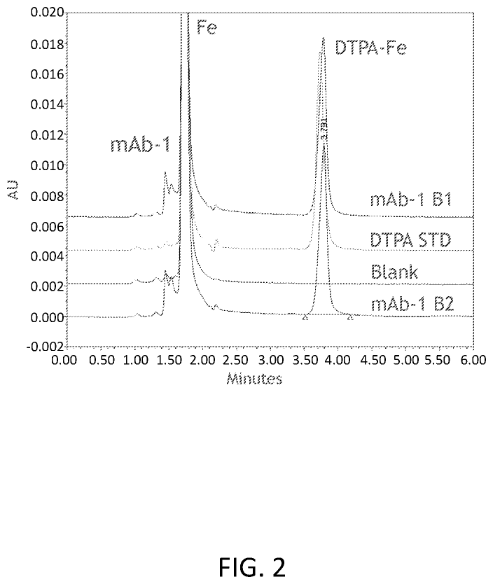

As further described herein, the methods of the present disclosure have certain distinct properties that are not shared by other methods for separating chelators and/or measuring the concentration of chelators known in the art. For example, the methods described herein allow for a one-step simple dilute and shoot of sample without separating the protein in the sample so that the chelator could be directly analyzed within a short period of time for analysis (see, e.g., FIG. 2). Furthermore, methods described herein allow for improved chelator separation and/or more accurate measurement of chelator concentration in a mixture compared to chromatography methods known in the art. Moreover, the chromatography column used herein maintains high performance after multiple injections. Overall, the methods described herein provide a robust and cost-effective way for analyzing the chelator present in a mixture containing protein, especially at high protein concentrations in biologics samples. Some aspects of the present disclosure are directed to methods of adjusting the concentration of a chelator of a composition comprising a protein, the method comprising measuring the concentration of a chelator present in the composition and adjusting the concentration of the chelator if it is higher or lower than a target concentration. Non-limiting examples of the various aspects are shown in the present disclosure.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

It is understood that wherever aspects are described herein with the language “comprising,” otherwise analogous aspects described in terms of “consisting of” and/or “consisting essentially of” are also provided.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. The headings provided herein are not limitations of the various aspects of the disclosure, which can be had by reference to the specification as a whole. Accordingly, the terms defined are more fully defined by reference to the specification in its entirety.

Abbreviations used herein are defined throughout the present disclosure. Various aspects of the disclosure are described in further detail in the following subsections.

The term “chelator” or “chelating agent,” as used interchangeably herein, refer to a molecule comprising nonmetal atoms, two or more of which atoms are capable of linking or binding with a metal ion to form a heterocyclic ring including the metal ion.

The term “chromatography,” as used herein, refers to a dynamic separation technique, which separates a target molecule such as a target protein (e.g., a charge variant of a protein, e.g., an antibody) from other molecules in the mixture (e.g., other charge variants) and allows it to be isolated. Typically, in a chromatography method, a liquid mobile phase transports a sample containing the target molecule of interest across or through a stationary phase (normally solid) medium. Differences in partition or affinity to the stationary phase causes the temporary binding of selected molecules to the stationary phase while the mobile phase carries different molecules out at different times.

The term “ion exchange chromatography,” as used herein, refers to a mode of chromatography where a target molecule, such as a protein (e.g., a charge variant of a protein) to be separated is isolated based on polar interactions with charged molecules (e.g., positively or negatively charged molecules) immobilized on the chromatography resin. Elution from an ion exchange chromatography column can be achieved using a salt gradient or changing the pH.

The term “high-performance liquid chromatography,” or “HPLC,” or “high-pressure liquid chromatography,” as used herein, refers to a chromatographic system that relies on pumps to pass a pressurized liquid and a sample mixture through a column filled with adsorbent, leading to the separation of the sample components. The components of the sample mixture are separated from each other due to their different degrees of interaction with the adsorbent particles.

The term “contacting,” as used herein, refers to applying a solution, e.g., a mixture comprising a protein and a chelator, as described herein, to a chromatography matrix. In some aspects, the term “contacting” is synonymous with “loading” or “injecting” a solution onto a chromatography column. A “column packing” or a “chromatography matrix” as used herein refers to the adsorbent solid material contained within a chromatography column.

The term “is applied to,” when used in the context of a gradient being applied to a chromatography matrix, broadly means that a gradient is formed, directly or indirectly, within and/or around a chromatography matrix. In some aspects, the chromatography matrix is present in a column, and the gradient is formed within the column. In some aspects, a gradient that is applied to a chromatography matrix is formed internally within a column, as opposed to a gradient which is formed externally and then added to a column. In some aspects, a gradient that is applied to the chromatography matrix forms within a column as a result of more than one buffer being added to the chromatography matrix. In some aspects, a gradient that is applied to the chromatography matrix is formed externally and then added to the column.

The term “void volume,” as used herein, refers specifically to the volume of the liquid phase contained inside a column. The void volume can indicate a portion of a column not occupied by a stationary phase. The void volume can be represented as being proportional to a diameter (e.g., inner diameter of the column) and length of the column.

The term “retained,” used in the context of the chelator being retained in a chromatography matrix, means that the chelator travels through the column more slowly than the velocity of an inert component that does not interact with the column packing material. The term “retention time” refers to the amount of time required for a component of a chemical mixture to pass through a chromatography column. The “retention time” equals to the time elapsed between sample injection and the maximum signal of the given chelator, protein, or metal at a chromatography detector, such as a UV-absorbance detector.

The term “peak response,” as used herein, refers to the peak area, which is the area under the curve of the detector signal trace as determined by chromatography. The peak area is proportional to the total quantity of the substance passing into the detector.

The term “standard” or “standard solution,” or “working standard solution” as used herein, refers to a solution containing a known concentration of a chelator. In some aspects, a series of standard solutions at different concentrations of a certain chelator is prepared to evaluate the linearity of UV absorbance versus the concentration of the chelator in the standard solution.

The term “diameter,” used in the context of the diameter of a protein, refers to the largest diameter of a protein, theoretically, the distance between the 2 atoms that are the farthest apart in the protein structure. The diameter of a protein can be determined by any suitable methods known in the art, e.g., dynamic light scattering (DLS).

The term “stability,” as used herein, refers to the physical, chemical, and conformational stability of an active molecular species in the composition according to the present disclosure. Stability can be measured as the length of time over which a molecular species, such as an antibody, in a formulation retains its original chemical identity or integrity. Instability of a protein formulation may be caused by chemical degradation or aggregation of the protein molecules to form higher order polymers, deglycosylation, modification of glycosylation, oxidation or any other structural modification that reduces at least one biological activity of a protein of the present invention.

The term “stable,” as used herein, refers to controlled degree of degradation, modification, aggregation, loss of biological activity and the like, of proteins.

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” can include a bispecific antibody or a multispecific antibody.

An “IgG antibody”, e.g., a human IgG1, IgG2, IgG3 and IgG4 antibody, as used herein has, in some aspects, the structure of a naturally-occurring IgG antibody, i.e., it has the same number of heavy and light chains and disulfide bonds as a naturally-occurring IgG antibody of the same subclass. For example, an IgG1, IgG2, IgG3 or IgG4 antibody can consist of two heavy chains (HCs) and two light chains (LCs), wherein the two HCs and LCs are linked by the same number and location of disulfide bridges that occur in naturally-occurring IgG1, IgG2, IgG3 and IgG4 antibodies, respectively (unless the antibody has been mutated to modify the disulfide bridges).

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the VL, VH, LC and CH1 domains; (ii) a F(ab′) 2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

As used herein, “isotype” refers to the antibody class (e.g., IgG1, IgG2, IgG3, IgG4, IgM, IgAQ1, IgA2, IgD, and IgE antibody) that is encoded by the heavy chain constant region genes.

As used herein, “heterologous moiety” refers to a polypeptide or polynucleotide is a polypeptide or polynucleotide that originates from a different protein or polynucleotide. The additional components of the fusion protein can originate from the same organism as the other polypeptide components of the fusion protein, or the additional components can be from a different organism than the other polypeptide components of the fusion protein. For instance, a heterologous polypeptide can be synthetic, or derived from a different species, different cell type of an individual, or the same or different type of cell of distinct individuals. In one aspect, a heterologous moiety is a polypeptide fused to another polypeptide to produce a fusion polypeptide or protein.

As used herein, the term “charge variant,” as used herein, refers to the full complement of product variant including, but not limited to acidic species, and basic species (e.g., Lys variants). In some aspects, such variants can include product aggregates and/or product fragments, to the extent that such aggregation and/or fragmentation results in a product charge variation as seen in an analytical technical technique used for that purpose.

### II. Methods of the Disclosure

A. Method of Separating a Chelator from a Protein Mixture

Disclosed herein are methods for separating a chelator in a mixture comprising a protein and the chelator. As demonstrated herein, in some aspects, such methods comprise contacting the mixture with a chromatography column which exhibits one or more properties that allow for the selective elution of the protein and the chelator from the mixture. Non-limiting examples of such properties include: (1) has a charge, which is the same as the charge of the protein; (2) is capable of forming a non-polar interaction with the chelator, (3) comprises a porous surface with a pore size that is less than the diameter of the protein, or (4) any combination of (1) to (3). Accordingly, in some aspects, a chromatography column useful for the present methods has a charge, which is the same as the charge of the protein. In some aspects, a chromatography column useful for the present methods is capable of forming a non-polar interaction with the chelator. In some aspects, a chromatography column that can be used with the present methods comprises a porous surface with a pore size that is less than the diameter of the protein. In some aspects, a chromatography column useful for the present methods (i) has a charge, which is the same as the charge of the protein, and (ii) is capable of forming a non-polar interaction with the chelator. In some aspects, a chromatography column useful for the present methods (i) has a charge, which is the same as the charge of the protein, and (ii) comprises a porous surface with a pore size that is less than the diameter of the protein. In some aspects, a chromatography column useful for the present methods (i) is capable of forming a non-polar interaction with the chelator, and (ii) comprises a porous surface with a pore size that is less than the diameter of the protein. In some aspects, a chromatography column that can be used with the present methods (i) has a charge, which is the same as the charge of the protein; (ii) is capable of forming a non-polar interaction with the chelator, and (iii) comprises a porous surface with a pore size that is less than the diameter of the protein.

As described elsewhere in the present disclosure, one of the properties of the chelator separation methods provided herein is that the methods allow for a one-step simple dilute and shoot of sample, which allows for a much quicker analysis for the chelator present in a mixture. Accordingly, in some aspects, the methods provided herein can comprise diluting the mixture (e.g., comprising a protein and the chelator) prior to contacting the mixture with the chromatography column. In some aspects, the mixture is diluted in a buffer (referred to herein as “dilution buffer”). Any suitable dilution buffers known in the art can be used to dilute the mixture comprising the protein and the chelator.

In some aspects, the dilution buffer comprises an acid addition salt. In some aspects, the acid addition salt is selected from organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like.

In some aspects, the dilution buffer comprises metal salts. A wide variety of metal salts can be employed including, for example, nitrates, iodides, chlorides, citrates, acetates and the like. The choice of an appropriate metal salt for any given metal as well as the choice of a particularly appropriate chelate for any given metal is within the skill of the art.

In some aspects, the dilution buffer comprises solvents that are commonly used in chromatography systems. In some aspects, the solvents is selected from ethyl acetate, chloroform, water, acetonitrile, isopropyl alcohol, methanol, ethanol, propanol, tetrahydrofuran, N,N-dimethylformamide, and dimethylsulfoxide.

In some aspects, the dilution buffer comprises metal ions. In some aspect, the metal ions are selected from zinc, nickel, copper, or iron (III).

Accordingly, in some aspects, the dilution buffer comprises an acid addition salt and a metal salt. In some aspects, the dilution buffer comprises an acid addition salt and a solvent. In some aspects, the dilution buffer comprises a solvent and a metal ion. In some aspects, the dilution buffer comprises an acid addition salt, metal salt, and a solvent. In some aspects, the dilution buffer comprises a metal salt, a solvent, and a metal ion. In some aspect, the dilution buffer comprises solvent, an acid addition salt, a metal salt, and a metal ion. In some aspects, the acid addition salt is selected from organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid, phosphoric acid, or the like, wherein the metal salts are selected from nitrates, iodides, chlorides, citrates, acetates and the like, wherein the metal ions are selected from zinc, nickel, copper, or iron (III), wherein the solvents is selected from ethyl acetate, chloroform, water, acetonitrile, isopropyl alcohol, methanol, ethanol, propanol, tetrahydrofuran, N,N-dimethylformamide, and dimethylsulfoxide.

In some aspects, the dilution buffer comprises acetonitrile, sulfuric acid, and iron (III) chloride (FeCl₃).

In some aspects, the acetonitrile is present in the buffer at a concentration of about 0.5%, about 1%, about 1.5%, about 2%, about 2.5%, or about 3%.

In some aspects, the sulfuric acid is present in the buffer at a concentration of about 0.01% to about 0.5%. In some aspects, the sulfuric acid is present in the buffer is at a concentration of about 0.05%, about 0.1%, about 0.15%, about 0.2%, about 0.25%, about 0.3%, about 0.35%, about 0.4%, about 0.45%, or about 0.5%. In some aspects, the sulfuric acid is present at a concentration of about 0.15% to about 0.20%.

In some aspects, the FeCl₃ is present in the buffer at a concentration of about 0.1 mM, about 0.2 mM, about 0.3 mM, about 0.4 mM, about 0.5 mM, about 0.6 mM, about 0.7 mM, about 0.8 mM, about 0.9 mM, or about 1.0 mM.

In some aspects, the dilution buffer comprises 2% Acetonitrile, 0.2% sulfuric acid, and 0.6 mM FeCl₃.

In some aspects, the dilution buffer comprises 2% Acetonitrile, 0.15% sulfuric acid, 0.6 mM FeCl₃.

In some aspects, the dilution buffer comprises 2% Acetonitrile, 0.1% sulfuric acid, and 0.6 mM FeCl₃.

In some aspects, the dilution buffer can have a pH of about 0.5 to about 6.0, or any pH in between. In some aspects, the dilution buffer can have a pH of about 0.5 to about 5.5, or any pH in between. In some aspects, the dilution buffer can have a pH of about 0.5 to about 5.0, or any pH in between. In some aspects, the dilution buffer can have a pH of about 0.5 to about 4.5, or any pH in between. In some aspects, the dilution buffer can have a pH of about 0.5 to about 4.0, or any pH in between. In some aspects, the dilution buffer has a pH of about 0.5. In some aspects, the dilution buffer has a pH of about 1.0. In some aspects, the dilution buffer has a pH of about 1.5. In some aspects, the dilution buffer has a pH of about 2.0. In some aspects, the dilution buffer has a pH of about 2.5. In some aspects, the dilution buffer has a pH of about 3.0. In some aspects, the dilution buffer has a pH of about 3.5. In some aspects, the dilution buffer has a pH of about 4.0. In some aspects, the dilution buffer has a pH of about 4.5. In some aspects, the dilution buffer has a pH of about 5.0. In some aspects, the dilution buffer has a pH of about 5.5. In some aspects, the dilution buffer has a pH of about 6.0.

In some aspects, the dilution buffer can have a pH of about 1.0 to about 6.0, or any pH in between. In some aspects, the dilution buffer can have a pH of about 1.0 to about 5.5, or any pH in between. In some aspects, the dilution buffer can have a pH of about 1.0 to about 5.0, or any pH in between. In some aspects, the dilution buffer can have a pH of about 1.0 to about 4.5, or any pH in between. In some aspects, the dilution buffer can have a pH of about 1.0 to about 4.0, or any pH in between. In some aspects, the dilution buffer can have a pH of about 1.0 to about 3.5, or any pH in between. In some aspects, the dilution buffer can have a pH of about 1.0 to about 3.0, or any pH in between. In some aspects, the dilution buffer can have a pH of about 1.0 to about 2.5, or any pH in between. In some aspects, the dilution buffer can have a pH of about 1.0 to about 2.0, or any pH in between. In some aspects, the dilution buffer can have a pH of about 1.0, about 1.5, about 2.0, about 2.5, about 3.0, about 3.5, about 4.0, about 4.5, about 5.0, about 5.5, or about 6.0. In some aspects, the dilution buffer can have a pH of about 1.0, about 2.0, about 3.0, or about 4.0.

In some aspects, the mixture is diluted in the dilution buffer at about 1:1 dilution, at about 1:2 dilution, at about 1:3 dilution, at about 1:4 dilution, at about 1:5 dilution, at about 1:6 dilution, at about 1:7 dilution, at about 1:8 dilution, at about 1:9 dilution, at about 1:10 dilution, at about 1:20 dilution, at about 1:30 dilution, at about 1:40 dilution, at about 1:50 dilution, at about 1:60 dilution, at about 1:70 dilution, at about 1:80 dilution, at about 1:90 dilution, at about 1:100 dilution, at about 1:200 dilution, at about 1:300 dilution, at about 1:400 dilution, or at about 1:500 dilution.

### B. Measuring the Concentration of the Chelator

As described and demonstrated herein, in some aspects, the methods provided above (e.g., separating a chelator in a mixture comprising a protein and the chelator), further comprises measuring the concentration of the chelator in the mixture. Not to be bound by any one theory, generally, the chelator separated by the chromatography techniques described herein can be quantified based on the elution profile and the chromatography conditions, because the amount of the chelator is proportional to the peak response of the chelator in the elution profile.

As further described and demonstrated herein, in some aspects, measuring the amount of chelator present in a mixture comprises quantifying the amount of chelator present in a standard solution. Accordingly, in some aspects, the method of measuring the amount of chelator present in a mixture provided herein further comprises contacting a standard solution with the chromatography column, wherein the standard solution comprises the chelator. The present disclosure allows a quick analysis of the amount of chelator present in a mixture because the chelator to be retained in the column in a relatively brief time period so that the whole process can proceed within a short time. In some aspects, the standard is retained within the chromatography column for at least about 2.0 minutes, at least about 2.5 minutes, at least about 3.0 minutes, at least about 3.5 minutes, at least about 4.0 minutes, at least about 4.5 minutes, or at least about 5.0 minutes. In some aspects, the standard is retained within the chromatography column for at least about 3.5 min. In some aspects, the standard is retained for about 3.0 minutes, for about 3.1 minutes, for about 3.2 minutes, for about 3.3 minutes, for about 3.4 minutes, for about 3.5 min, for about 3.6 min, for about 3.7 min, for about 3.8 min, for about 3.9 min, for about 4.0 min, for about 4.1 minutes, for about 4.2 minutes, for about 4.3 minutes, for about 4.4 minutes, or for about 4.5 minutes. In some aspects, the standard is retained within the chromatography column for between about 3.5 minutes to about 4.1 minutes.

In some aspects, the method of measuring the concentration of the chelator in the mixture comprises (i) obtaining a peak response from the mixture (rᵤ), (ii) obtaining a peak response from the standard solution (rₛ), (iii) or both (i) and (ii), wherein the peak response is the peak area as determined by a chromatography detector known in the art. The most common liquid chromatography detectors are ultraviolet detector, the fluorescence detector, and the refractometer. In some aspects, the peak area is determined by high performance liquid chromatography (“HPLC”) at the wavelength of 260 nm using an ultraviolet detector. In some aspects, the method of measuring the concentration of the chelator in the mixture comprises obtaining a peak response from the mixture (rᵤ), wherein the peak response is the peak area as determined by high performance liquid chromatography (“HPLC”) at the wavelength of 260 nm. In some aspects, the method of measuring the concentration of the chelator in the mixture comprises obtaining a peak response from the standard solution (rₛ), wherein the peak response is the peak area as determined by high performance liquid chromatography (“HPLC”) at the wavelength of 260 nm. In some aspects, the method of measuring the concentration of the chelator in the mixture comprises both obtaining a peak response from the mixture (rᵤ) and obtaining a peak response from the standard solution (rₛ), wherein the peak response is the peak area as determined by high performance liquid chromatography (“HPLC”) at the wavelength of 260 nm.

In some aspects, the concentration of the chelator is calculated as formula (I):

$$C ( {V}_{u} / {V}_{n} ) ( {r}_{u} / {r}_{s} )$$

wherein C is the concentration (μg/mL) of the chelator in the standard solution; rᵤ and rₛ are the peak responses obtained from the mixture and the standard solution, respectively, Vᵤ is the total volume (μL) of the mixture after being diluted; Vₙ is the volume (μL) of the mixture before the dilution,
wherein C is calculated as formula (II):

$$[ {W}_{s} \times P ] \times D \times 1000 / {V}_{s}$$

wherein Ws is the weight (mg) of a reference material for the chelator, P is the purity of the reference material (expressed as a fraction), Vs is the volume of the standard solution (mL), and D is the Dilution Factor (1/100).

### C. Method of Adjusting Chelator Concentration in a Composition

Because chelators are routinely used in biologics compositions to prevent oxidation induced by metal ions, a rapid and reliable method for monitoring chelator concentrations during formulation and process development allows adjusting the concentration of chelators when needed to increase or decrease the concentration of chelator in a mixture, such as a biologics composition being tested. Therefore, in some aspects, disclosed herein is a method comprising measuring the concentration of a chelator present in a composition and adjusting the concentration of the chelator to a desired concentration according to the methods described above.

In some aspects, the method of adjusting the chelator concentration in a composition comprises adjusting the concentration of the chelator present in the composition based on a reference amount (e.g., the concentration of the chelator present in a standard solution; also referred to herein as the “target concentration” or “target amount”). In some aspects, the method of adjusting the chelator concentration of a composition comprises increasing the concentration of the chelator if the measured concentration of the chelator is less than a reference amount (e.g., the concentration of the chelator present in a standard solution). In some aspects, increasing the concentration of the chelator comprises adding an amount of the chelator to the composition. In some aspects, the concentration of the chelator is measured after increasing the concentration of the chelator present in the composition (e.g., adding an amount of the chelator to the composition).

In some aspects, after increasing the concentration of the chelator (e.g. adding the amount of the chelator to the composition), the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference amount.

In some aspects, the method of adjusting the chelator concentration of a composition comprises decreasing the concentration of the chelator if the measured concentration of the chelator is higher than a reference amount (e.g., the concentration of the chelator present in a standard solution). In some aspects, decreasing the concentration of the chelator comprises adding a diluent to the composition, increasing the concentration of the protein in the composition, or both. In some aspects, decreasing the concentration of the chelator comprises adding a diluent (e.g., dilution buffer) to the composition. In some aspects, decreasing the concentration of the chelator comprises increasing the concentration of the protein in the composition (e.g., by adding additional amount of the protein to the composition). In some aspects, decreasing the concentration of the chelator comprises both adding a diluent to the composition and increasing the concentration of the protein in the composition. In some aspects, the concentration of the chelator is measured after decreasing the concentration of the chelator present in the composition (e.g., adding a diluent to the composition, increasing the concentration of the protein in the composition, or both).

In some aspects, after decreasing the concentration of the chelator, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference amount.

As is apparent from the above disclosure, some aspects of the present disclosure are related to a method of producing a composition comprising a protein, such as an antibody, wherein the method comprises measuring the concentration of a chelator present in the composition according to any of the methods described herein. In some aspects, the method further comprises adjusting the concentration of the chelator present in the composition according to any of the methods provided herein (e.g., described above).

In some aspects, the method of adjusting the chelator concentration of a composition comprises increasing the concentration of the chelator if the measured concentration of the chelator is less than a reference amount (e.g., the concentration of the chelator present in a standard solution). In some aspects, increasing the concentration of the chelator comprises adding an amount of the chelator to the composition. In some aspects, the concentration of the chelator is measured again after adding an amount of the chelator to the composition. Accordingly, in some aspects, increasing the chelator concentration of a composition can comprise multiple measuring steps. In some aspects, the multiple measuring steps can be followed by the addition of an amount of the chelator to the composition if the measured concentration of the chelator is less than the reference amount.

In some aspects, after adding the amount of the chelator to the composition, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference.

In some aspects, the method of adjusting the chelator concentration of a composition comprises decreasing the concentration of the chelator if the measured concentration of the chelator is higher than a reference amount (e.g., the concentration of the chelator present in a standard solution). In some aspects, decreasing the concentration of the chelator comprises adding a diluent to the composition, increasing the concentration of the protein in the composition, or both. In some aspects, the concentration of the chelator is measured again after adding a diluent to the composition, increasing the concentration of the protein in the composition, or both. Accordingly, in some aspects, decreasing the chelator concentration of a composition can comprise multiple measuring steps. In some aspects, the multiple measuring steps can be followed by a step in which the concentration of the chelator is decreased (e.g., by adding a diluent to the composition, increasing the concentration of the protein in the composition, or both).

In some aspects, after adding a diluent to the composition, increasing the concentration of the protein in the composition, or both, the measured concentration of the chelator is greater than about 5%, greater than about 10%, greater than about 20%, greater than about 30%, greater than about 40%, greater than about 50%, greater than about 60%, greater than about 70%, greater than about 80%, greater than about 90%, greater than about 100%, greater than about 125%, greater than about 150%, greater than about 175%, or greater than about 200% as compared to the reference amount.

In some aspects, after decreasing the concentration of the chelator, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference.

### D. Column Chromatography

### Criteria for Column Selection

As described herein, some aspects of the methods of the present disclosure comprise contacting a mixture comprising a chelator and a protein to a chromatography column. As is apparent from the present disclosure, chromatography columns suitable for the methods provided herein exhibit one or more of the properties that allow for the selective elution of a protein and chelator from a mixture, e.g., (1) has a charge, which is the same as the charge of the protein; (2) is capable of forming a non-polar interaction with the chelator, and (3) comprises a porous surface with a pore size that is less than the diameter of the protein, or (4) any combination of (1) to (3). Additional aspects of suitable chromatography columns are provided below (and as well as throughout the present application).

In some aspects, the chromatography column has the same charge as the protein present in the mixture, which is contacted to the chromatography column. In some aspects, both the chromatography column and the protein have a positive charge. In some aspects, the chromatography column has a charge that differs from other molecules (e.g., chelator) present in the mixture. Accordingly, as will be apparent to those skilled in the arts, where a mixture comprising both a protein (e.g., positively charged) and other molecules (e.g., negatively charged) is contacted with a chromatography column (e.g., positively charged), in some aspects, the protein passes through the chromatography column much more quickly compared to other molecules (e.g., chelators) present in the mixture. In some aspects, the chromatography column is capable of forming a non-polar interaction with the chelator that has formed complex with metal ions.

As described herein, in some aspects, the pore size of the chromatography column is less than the diameter of the protein in the mixture. In some aspects, the pore size of the chromatography column is less than about 99%, less than about 98%, less than about 97%, less than about 96%, less than about 95%, less than about 94%, less than about 93%, less than about 92%, less than about 91%, less than about 90%, less than about 85%, less than about 80%, less than about 75%, less than about 70%, less than about 65%, less than about 60%, less than about 55%, or less than about 50% of the diameter of the protein. Not to be bound by any one theory and as will be apparent to those skilled in the arts, in some aspects, because the diameter of the protein is larger than the pore size of the chromatography column, the protein can readily pass through the chromatography column. Accordingly, in some aspects, the protein can pass through the chromatography column much more quickly compared to other molecules (which have a diameter smaller than the pore size, e.g., chelator) present in the mixture.

In some aspects, the pore size of the chromatography column is no more than about 150 Å. In some aspects, the pore size of the chromatography column is no more than about 140 Å. In some aspects, the pore size is no more than about 130 Å. In some aspects, the pore size is no more than about 120 Å. In some aspects, the pore size of the chromatography column is no more than about 110 Å. For instance, in some aspects, the pore size of the chromatography column is about 110 Å, about 100 Å, about 90 Å, about 80 Å, or about 70 Å. In some aspects, the pore size of the chromatography column is about 100 Å.

As demonstrated herein, in some aspects, the methods provided herein can be performed using liquid chromatography. In some aspects, the liquid chromatography comprises a high performance liquid chromatography (HPLC) or ultra performance liquid chromatography (UPLC). Accordingly, in some aspects, the chromatography column is a hydrophilic interaction liquid chromatography (HILIC) column.

### The Stationary Phase of Chromatography

In any chromatography (including those in the mixture), generally, there are two primary phases: (1) a stationary phase and (2) a mobile phase. As used herein, the term “stationary phase” refers to a phase that comprises particles that comprise an organic or an inorganic material that optionally has an organic moiety bonded to it that renders the surface of the particle useful in certain chromatographic separations. In certain instances, these particles or materials are fixed in a column and do not move. As used herein, the term “mobile phase” refers to the fluid phase (generally a liquid) that flows through the chromatographic column. For instance, in any of the methods provided herein, the “mobile phase” can refer to the mixture that comprises the protein and is passed through the chromatography column. As is apparent from the present disclosure, in some aspects, by modifying different variables of the stationary and/or mobile phase can adjust the overall efficiency of the methods provided herein.

The stationary phase (e.g., HPLC column) can be a resin or media suitable for separation of chelator from a mixture containing protein using the methods described herein. Non-limiting examples of the stationary phase used can include silica, amide, aminopropyl, diol, zwitterionic (for example, sulfoalkylbetaine) phases, bonded phases upon silica, or bonded phases on organic polymer matrices. In some aspects, the stationary phase is silica.

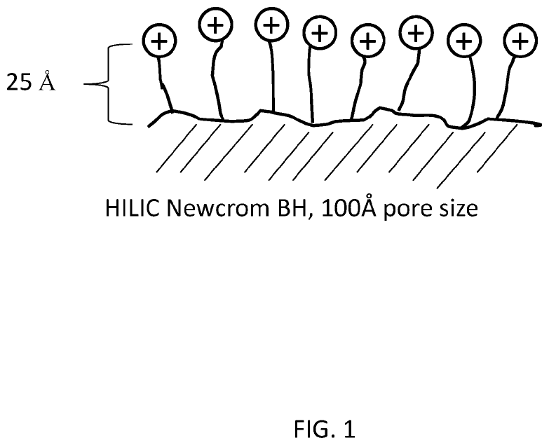

In some aspects, the stationary phase of the chromatography column comprises a silica gel surface, wherein a hydrophobic ligand is attached to the silica gel surface, and a charged functional group is attached to the terminal end of the chain (see, e.g., FIG. 1). As a result, the positive charged terminal functional group has high mobility inside the pores of the stationary phase. Not to be bound by any one theory, in some aspects, this can prevent the chelator from being trapped in the column. In some aspects, the positive charged terminal functional group comprises an amino group (such as primary, secondary, tertiary or quaternary amines). In some aspects, the positively charged terminal functional group comprises an amine group. In some aspects, the positively charged terminal functional group comprises dimethylaminopropyl. In some aspects, the positively charged terminal functional group comprises polyethyleneimine.

In some aspects, the hydrophobic ligand comprises an alkyl chain. In some aspects, the hydrophobic ligand is from about 10 Å to about 50 Å. In some aspects, the hydrophobic ligand is an alkyl chain, wherein the length of the alkyl chain is from about 10 Å to about 50 Å. In some aspects, the length of the hydrophobic ligand (e.g., alkyl chain) is about 10 Å, about 15 Å, about 20 Å, about 25 Å, about 30 Å, about 35 Å, about 40 Å, about 45 Å, or about 50 Å. In some aspects, the length of the hydrophobic ligand (e.g., alkyl chain) is about 10 Å. In some aspects, the length of the hydrophobic ligand (e.g., alkyl chain) is about 15 Å. In some aspects, the length of the hydrophobic ligand (e.g., alkyl chain) is about 20 Å. In some aspects, the length of the hydrophobic ligand (e.g., alkyl chain) is about 25 Å. In some aspects, the length of the hydrophobic ligand (e.g., alkyl chain) is about 35 Å. In some aspects, the length of the hydrophobic ligand (e.g., alkyl chain) is about 45 Å. In some aspects, the length of the hydrophobic ligand (e.g., alkyl chain) is about 50 Å.

As is apparent from the present disclosure, suitable chromatography columns that can be used with the methods provided herein are available in the art. Non-limiting examples of such columns include: Newcrom BH column and Newcrom B column.

### The Mobile Phase of Chromatography

Mobile phase of chromatography typically carries the components in the mixture described herein through or across the chromatography column described herein. In some aspects, the mobile phase of the chromatography comprises solvents that are commonly used in chromatography systems. Non-limiting examples of such solvent include ethyl acetate, chloroform, water, acetonitrile, isopropyl alcohol, methanol, ethanol, propanol, tetrahydrofuran, N,N-dimethylformamide, and dimethylsulfoxide. A typical mobile phase used in HPLC comprises water and an organic solvent. In some aspects, the mobile phase of the chromatography comprises the dilution buffer described herein. As is apparent from the present disclosure, in some aspects, the mobile phase comprises the chelator and/or proteins, such as those present in the mixture that is contacted to the chromatography column. Provided below are additional aspects of chelators and/or proteins that can be present in the mobile phase.

### Chelators

In some aspects, the chelator in the mixture can be any metal ion chelator. The term “chelator,” as used herein, refers to a chelator free of metal ion or a complex of a chelator with a metal ion. In some aspects, the chelator is selected from polyaminocarboxylate acids, hydroxyaminocarboxylic acids, N-substituted glycines, 2-(2-amino-2-oxoethyl)aminoethane sulfonic acid (BES), deferoxamine (DEF), citric acid, niacinamide, desoxycholates or combinations thereof.

In some aspects, the chelator is a polyaminocarboxylate acid. In some aspects, the polyaminocarboxylate acid is selected from DTPA, NTA, EDTA, ethylenediaminediacetate (HDDA), ethyl-enebis(oxyethylenenitrilo) tetraacetate (EGTA)N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, aspartic acid, or a combination thereof.

In some aspects, the chelator is a hydroxyaminocarboxylic acid. In some aspects, the hydroxyaminocarboxylic acid is selected from N-hydroxyethyliminodiacetic acid (HIMDA); N,N-bis-hydroxyethylglycine (bicine); N-(trishydroxymethylmethyl) 10 glycine (tricine); or combinations thereof.

In some aspects, the chelator is a N-substituted glycine. In some aspect, the N-substituted glycine is glycylglycine.

In some aspects, the chelator is BES.

In some aspects, the chelator is DEF.

In some aspects, the chelator is a citric acid.

In some aspects, the chelator is a niacinamide.

In some aspects, the chelator is a desoxycholate. In some aspects, the desoxycholate is sodium desoxycholate. It is contemplated that any chelator which binds barium, calcium, cerium, cobalt, copper, iron, magnesium, manganese, nickel, strontiurn, or zinc could be quantified using the methods provided herein.

In some aspects, a mixture comprises a single chelator. In some aspects, a mixture comprises multiple chelators. In some aspects, each of the multiple chelators present in the mixture are different. In some aspects, the chelator in the mixture comprises DTPA, EDTA, or both.

In some aspects, the chelator in the mixture is DTPA.

### Proteins/Polypeptides

In some aspects, the mixture or composition to be analyzed described herein comprises one or more polypeptides. In some aspects, the polypeptide is a protein. In some aspects, the protein/polypeptide present in the mixture is a charge variant of the protein (also referred to herein as “species”). In some aspects, the species is an acidic species. In some aspects, the species is a basic species. In some aspects, the species is the main species.

As described elsewhere in the present disclosure, in some aspects, the diameter of the protein is larger than the pore size of the chromatography column. In some aspects, the diameter of the protein in the mixture is less than about 80 Å, less than about 90 Å, less than about 100 Å, less than about 110 Å, less than about 120 Å, less than about 130 Å, less than about 140 Å, less than about 150 Å, less than about 160 Å, less than about 170 Å, or less than about 180 Å. In some aspects, the diameter of the protein is less than about 70 Å. In some aspects, the diameter of the protein is less than about 80 Å. In some aspects, the diameter of the protein is less than about 90 Å. In some aspects, the diameter of the protein is less than about 100 Å. In some aspects, the diameter of the protein is less than about 110 Å. In some aspects, the diameter of the protein is less than about 120 Å. In some aspects, the diameter of the protein is less than about 130 Å. In some aspects, the diameter of the protein is less than about 140 Å. In some aspects, the diameter of the protein is less than about 150 Å. In some aspects, the diameter of the protein is less than about 160 Å. In some aspects, the diameter of the protein is less than about 170 Å. In some aspects, the diameter of the protein is less than about 180 Å.

In some aspects, the diameter of the protein in the mixture is between about 70 Å and about 180 Å, between about 70 Å and about 170 Å, between about 70 Å and about 160 Å, between about 70 Å and about 150 Å, between 70 about Å and about 140 Å, between about 70 Å and about 130 Å, between about 70 Å and about 120 Å, between about 70 Å and about 110 Å, between about 70 Å and about 100 Å, between about 80 Å and about 180 Å, between about 80 Å and about 170 Å, between about 80 Å and about 160 Å, between about 80 Å and about 150 Å, between about 80 Å and about 140 Å, between about 80 Å and about 130 Å, between about 80 Å and about 120 Å, between about 80 Å and about 110 Å, between about 80 Å and about 100 Å, between about 90 Å and about 180 Å, between about 90 Å and about 170 Å, between about 90 Å and about 160 Å, between about 90 Å and about 150 Å, between about 90 Å and about 140 Å, between about 90 Å and about 130 Å, between about 90 Å and about 120 Å, between about 90 Å and about 110 Å, between about 90 Å and about 100 Å, between about 100 Å and about 180 Å, between about 100 Å and about 170 Å, between about 100 Å and about 160 Å, between about 100 Å and about 150 Å, between about 100 Å and about 140 Å, between about 100 Å and about 130 Å, between about 100 Å and about 120 Å, between about 100 Å and about 110 Å, between about 110 Å and about 180 Å, between about 110 Å and about 170 Å, between about 110 Å and about 160 Å, between about 110 Å and about 150 Å, between about 110 Å and about 140 Å, between about 110 Å and about 130 Å, between about 110 Å and about 120 Å, between about 120 Å and about 180 Å, between about 120 Å and about 170 Å, between about 120 Å and about 160 Å, between about 120 Å and about 150 Å, between about 120 Å and about 140 Å, between about 120 Å and about 130 Å, between about 130 Å and about 180 Å, between about 130 Å and about 170 Å, between about 130 Å and about 160 Å, between about 130 Å and about 150 Å, between about 130 Å and about 140 Å, between about 140 Å and about 180 Å, between about 140 Å and about 170 Å, between about 140 Å and about 160 Å, between about 140 Å and about 150 Å, between about 150 Å and about 180 Å, between about 150 Å and about 170 Å, between about 150 Å and about 160 Å, between about 160 Å and about 180 Å, between about 160 Å and about 170 Å, or between about 170 Å and about 180 Å.

In some aspects, the diameter of the protein in the mixture is between about 70 Å and about 180 Å. In some aspects, the diameter of the protein in the mixture is between about 70 Å and about 170 Å. In some aspects, the diameter of the protein in the mixture is between about 70 Å and about 160 Å. In some aspects, the diameter of the protein in the mixture is between about 70 Å and about 150 Å. In some aspects, the diameter of the protein in the mixture is between 70 about Å and about 140 Å. In some aspects, the diameter of the protein in the mixture is between about 70 Å and about 130 Å. In some aspects, the diameter of the protein in the mixture is between about 70 Å and about 120 Å. In some aspects, the diameter of the protein in the mixture is between about 70 Å and about 110 Å. In some aspects, the diameter of the protein in the mixture is between about 70 Å and about 100 Å. In some aspects, the diameter of the protein in the mixture is between about 80 Å and about 180 Å. In some aspects, the diameter of the protein in the mixture is between about 80 Å and about 170 Å. In some aspects, the diameter of the protein in the mixture is between about 80 Å and about 160 Å. In some aspects, the diameter of the protein in the mixture is between about 80 Å and about 150 Å. In some aspects, the diameter of the protein in the mixture is between about 80 Å and about 140 Å. In some aspects, the diameter of the protein in the mixture is between about 80 Å and about 130 Å. In some aspects, the diameter of the protein in the mixture is between about 80 Å and about 120 Å. In some aspects, the diameter of the protein in the mixture is between about 80 Å and about 110 Å. In some aspects, the diameter of the protein in the mixture is between about 80 Å and about 100 Å. In some aspects, the diameter of the protein in the mixture is between about 90 Å and about 180 Å. In some aspects, the diameter of the protein in the mixture is between about 90 Å and about 170 Å. In some aspects, the diameter of the protein in the mixture is between about 90 Å and about 160 Å. In some aspects, the diameter of the protein in the mixture is between about 90 Å and about 150 Å. In some aspects, the diameter of the protein in the mixture is between about 90 Å and about 140 Å. In some aspects, the diameter of the protein in the mixture is between about 90 Å and about 130 Å. In some aspects, the diameter of the protein in the mixture is between about 90 Å and about 120 Å. In some aspects, the diameter of the protein in the mixture is between about 90 Å and about 110 Å. In some aspects, the diameter of the protein in the mixture is between about 90 Å and about 100 Å. In some aspects, the diameter of the protein in the mixture is between about 100 Å and about 180 Å. In some aspects, the diameter of the protein in the mixture is between about 100 Å and about 170 Å. In some aspects, the diameter of the protein in the mixture is between about 100 Å and about 160 Å. In some aspects, the diameter of the protein in the mixture is between about 100 Å and about 150 Å. In some aspects, the diameter of the protein in the mixture is between about 100 Å and about 140 Å. In some aspects, the diameter of the protein in the mixture is between about 100 Å and about 130 Å. In some aspects, the diameter of the protein in the mixture is between about 100 Å and about 120 Å. In some aspects, the diameter of the protein in the mixture is between about 100 Å and about 110 Å. In some aspects, the diameter of the protein in the mixture is between about 110 Å and about 180 Å. In some aspects, the diameter of the protein in the mixture is between about 110 Å and about 170 Å. In some aspects, the diameter of the protein in the mixture is between about 110 Å and about 160 Å. In some aspects, the diameter of the protein in the mixture is between about 110 Å and about 150 Å. In some aspects, the diameter of the protein in the mixture is between about 110 Å and about 140 Å. In some aspects, the diameter of the protein in the mixture is between about 110 Å and about 130 Å. In some aspects, the diameter of the protein in the mixture is between about 110 Å and about 120 Å. In some aspects, the diameter of the protein in the mixture is between about 120 Å and about 180 Å. In some aspects, the diameter of the protein in the mixture is between about 120 Å and about 170 Å. In some aspects, the diameter of the protein in the mixture is between about 120 Å and about 160 Å. In some aspects, the diameter of the protein in the mixture is between about 120 Å and about 150 Å. In some aspects, the diameter of the protein in the mixture is between about 120 Å and about 140 Å. In some aspects, the diameter of the protein in the mixture is between about 120 Å and about 130 Å. In some aspects, the diameter of the protein in the mixture is between about 130 Å and about 180 Å. In some aspects, the diameter of the protein in the mixture is between about 130 Å and about 170 Å. In some aspects, the diameter of the protein in the mixture is between about 130 Å and about 160 Å. In some aspects, the diameter of the protein in the mixture is between about 130 Å and about 150 Å. In some aspects, the diameter of the protein in the mixture is between about 130 Å and about 140 Å. In some aspects, the diameter of the protein in the mixture is between about 140 Å and about 180 Å. In some aspects, the diameter of the protein in the mixture is between about 140 Å and about 170 Å. In some aspects, the diameter of the protein in the mixture is between about 140 Å and about 160 Å. In some aspects, the diameter of the protein in the mixture is between about 140 Å and about 150 Å. In some aspects, the diameter of the protein in the mixture is between about 150 Å and about 180 Å. In some aspects, the diameter of the protein in the mixture is between about 150 Å and about 170 Å. In some aspects, the diameter of the protein in the mixture is between about 150 Å and about 160 Å. In some aspects, the diameter of the protein in the mixture is between about 160 Å and about 180 Å. In some aspects, the diameter of the protein in the mixture is between about 160 Å and about 170 Å. In some aspects, the diameter of the protein in the mixture is between about 170 Å and about 180 Å.

In some aspects, the concentration of the protein in the mixture is about 30 mg/mL to about 300 mg/mL. In some aspects, the concentration of the protein in the mixture is about 30 mg/mL to about 100 mg/mL, about 50 mg/mL to about 120 mg/mL, about 70 mg/mL to about 150 mg/mL, about 100 mg/mL to about 200 mg/mL, about 150 mg/mL to about 250 mg/mL, or about 200 mg/mL to about 300 mg/mL.

In some aspects, the concentration of the protein in the mixture is about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, or about 300 mg/mL. In some aspects, the concentration of the protein in the mixture is about 30 mg/mL. In some aspects, the concentration of the protein in the mixture is about 40 mg/mL. In some aspects, the concentration of the protein in the mixture is about 50 mg/mL. In some aspects, the concentration of the protein in the mixture is about 60 mg/mL. In some aspects, the concentration of the protein in the mixture is about 70 mg/mL. In some aspects, the concentration of the protein in the mixture is about 80 mg/mL. In some aspects, the concentration of the protein in the mixture is about 90 mg/mL. In some aspects, the concentration of the protein in the mixture is about 100 mg/mL. In some aspects, the concentration of the protein in the mixture is about 110 mg/mL. In some aspects, the concentration of the protein in the mixture is about 120 mg/mL. In some aspects, the concentration of the protein in the mixture is about 130 mg/mL. In some aspects, the concentration of the protein in the mixture is about 140 mg/mL. In some aspects, the concentration of the protein in the mixture is about 150 mg/mL. In some aspects, the concentration of the protein in the mixture is about 160 mg/mL. In some aspects, the concentration of the protein in the mixture is about 170 mg/mL. In some aspects, the concentration of the protein in the mixture is about 180 mg/mL. In some aspects, the concentration of the protein in the mixture is about 190 mg/mL. In some aspects, the concentration of the protein in the mixture is about 200 mg/mL. In some aspects, the concentration of the protein in the mixture is about 210 mg/mL. In some aspects, the concentration of the protein in the mixture is about 220 mg/mL. In some aspects, the concentration of the protein in the mixture is about 230 mg/mL. In some aspects, the concentration of the protein in the mixture is about 240 mg/mL. In some aspects, the concentration of the protein in the mixture is about 250 mg/mL. In some aspects, the concentration of the protein in the mixture is about 260 mg/mL. In some aspects, the concentration of the protein in the mixture is about 270 mg/mL. In some aspects, the concentration of the protein in the mixture is about 280 mg/mL. In some aspects, the concentration of the protein in the mixture is about 290 mg/mL. In some aspects, the concentration of the protein in the mixture is about 300 mg/mL.

In some aspects, the protein is conjugated to a drug, such that the mixture of the composition comprises a protein-drug conjugate (PDC). In some aspects, the protein-drug conjugate comprises an antibody-drug conjugate (ADC).

In some aspects, the protein comprises a fusion protein. In some aspects, the fusion protein further comprises a heterologous moiety. In some aspects, the heterologous moiety is a half-life extending moiety. In some aspects, the heterologous moiety comprises a non-polypeptide moiety. In some aspects, the heterologous moiety comprises a polypeptide. In some aspects, the heterologous moiety comprises albumin, an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof.

In some aspects, the protein comprises an immunoglobulin fused to a growth factor, a cytokine, a chemokine, an enzyme, a hormone, or any combination thereof. In some aspects, the protein comprises an Fc fused to an interleukin.

In some aspects, the protein comprises an antibody or an antigen binding portion thereof. In some aspects, the antibody or antigen binding portion thereof binds an antigen selected from CD40, CD70, CD96, CXCR7, ICOS, OX40, PD-L1, TLR9, and any combination thereof.

In some aspects, the antibody or antigen-binding portion thereof specifically binds CD40. Various monoclonal antibodies that bind specifically to CD40 have been described in WO 2014/065403, WO 2003/029296, WO 2021/222188, WO 2016/196314, WO 2018/222019, WO 2016/028810, WO 2003/040170, WO 2006/073443, and WO 2022/061061, each of which is incorporated by reference in its entirety.

In some aspects, the antibody or antigen-binding portion thereof specifically binds CD70. Various monoclonal antibodies that bind specifically to CD70 have been described in WO 2021/245603, WO 2006/044643, WO 2019/152705, WO 2022/002019, WO 2006/113909, and WO 2013/138586, each of which is incorporated by reference in its entirety.

In some aspects, the antibody or antigen-binding portion thereof specifically binds CD96. Various monoclonal antibodies that bind specifically to CD96 have been described in WO 2019/091449, WO 2020/132034, and WO 2021/042019, each of which is incorporated by reference in its entirety.

In some aspects, the antibody or antigen-binding portion thereof specifically binds CXCR7. Various monoclonal antibodies that bind specifically to CXCR7 have been described in WO 2008/048519 and WO 2010/141986, each of which is incorporated by reference in its entirety. In some aspects, the antibody or antigen-binding portion thereof specifically binds TLR9. Various monoclonal antibodies that bind specifically to TLR9 have been described in WO 2020/068557 and WO 2004/096156, each of which is incorporated by reference in its entirety.

In some aspects, the antibody or antigen-binding portion thereof specifically binds PD-L1. In certain aspects, the anti-PD-L1 antibody is selected from the group consisting of atezolizumab (Roche; also known as TECENTRIQ®; MPDL3280A, RG7446; see U.S. Pat. No. 8,217,149; see, also, Herbst et al. (2013) J Clin Oncol 31 (suppl): 3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl): TPS3113 (2018)), and CK-301 (Checkpoint Therapeutics; see Gorelik et al., AACR: Abstract 4606 (April 2016)).

In certain aspects, the PD-L1 antibody is atezolizumab (TECENTRIQ®). In certain aspects, the PD-L1 antibody is durvalumab (IMFINZI™). In certain aspects, the PD-L1 antibody is avelumab (BAVENCIO®).

In some aspects, the antibody or antigen-binding portion thereof specifically binds OX40 (also known as CD134, TNFRSF4, ACT35 and/or TXGP1L). In some aspects, the anti-OX40 antibody is BMS-986178 (Bristol-Myers Squibb Company), described in Int'l Publ. No. WO20160196228. In some aspects, the anti-OX40 antibody is selected from the anti-OX40 antibodies described in Int'l Publ. Nos. WO95012673, WO199942585, WO14148895, WO15153513, WO15153514, WO13038191, WO16057667, WO03106498, WO12027328, WO13028231, WO16200836, WO 17063162, WO17134292, WO 17096179, WO 17096281, and WO 17096182, each of which is incorporated by reference herein in its entirety.

In some aspects, the antibody or antigen-binding portion thereof specifically binds ICOS In some aspects, the anti-ICOS antibody is selected from anti-ICOS antibodies described in, for example, WO 2016/154177 (Jounce Therapeutics, Inc.), WO 2008/137915 (MedImmune), WO 2012/131004 (INSERM, French National Institute of Health and Medical Research), EP3147297 (INSERM, French National Institute of Health and Medical Research), WO 2011/041613 (Memorial Sloan Kettering Cancer Center), EP 2482849 (Memorial Sloan Kettering Cancer Center), WO 1999/15553 (Robert Koch Institute), U.S. Pat. Nos. 7,259,247 and 7,722,872 (Robert Kotch Institute); WO 1998/038216 (Japan Tobacco Inc.), U.S. Pat. Nos. 7,045,615; 7,112,655, and 8,389,690 (Japan Tobacco Inc.), U.S. Pat. Nos. 9,738,718 and 9,771,424 (GlaxoSmithKline), and WO 2017/220988 (Kymab Limited), each of which is incorporated by reference herein in its entirety.

Various aspects of the disclosure are described in further detail in the following subsections. The present disclosure is further illustrated by the following examples which should not be construed as further limiting.

### Examples

### Example 1: Materials and Methods

The Examples described below use one or more of the following materials and methods.

### Equipment

For chromatographic separations, the following equipment were used: The HPLC system was designed for analytical separations and paired with a fraction collector using UV detection.

Where appropriate, the following equipment was also used: Waters Alliance system equipped with a Waters Model 2487 UV/Vis Detector.

### Chemicals and Materials

Water is distilled or deionized water, further purified using a Millipore Milli-Q or equivalent. All other reagents were purchased from Sigma-Aldrich (St. Louis, MO, USA) unless otherwise specified.

### HPLC Parameters

The chromatography column used was Sielc Newcrom BH, 150 mm×3.2 mm i.d., 3 μm particle size. The Detector Wavelength was 260 nm. Column temperature was 40° C. The injection volume was 20 μL. The flow rate was 0.5 mL/minute, and the running time of the program was 5 minutes.

### Standard and Sample Preparations

DTPA Stock Standard Solution (0.4 mg/mL): Transfer 40±3 mg of accurately weighed DPTA reference material into a 100-mL volumetric flask. Add ˜90 mL of water. Sonicate for 30 minutes or until dissolved. If necessary, vortex the solution to aid dissolution. Bring up to volume with water and mix well. Solution could be used for up to 30 days when stored at 2-8° C.

DTPA Working Stock Standard Solution (20 μg/mL): Transfer 1.0 mL of DPTA Stock Standard Solution into a 20-mL volumetric flask. Bring up to volume with water and mix well. Solution can be used for up to 30 days when stored at 2-8° C.

DTPA Working Standard Solution (4 μg/mL, System Suitability Solution): Accurately transfer 200 μL of DPTA Working Stock Standard Solution into an HPLC vial. Add 800 μL diluent. Vertex for up to 30 seconds to mix well for HPLC analysis. Solution can be used for up to 48 hours when stored at 2-8° C. Prepare enough vials for system equilibration and system suitability tests.

Sample Preparation for Assay of DTPA in mAb Drug Substance and Drug Product: Use calibrated positive displacement pipettes to dilute mAb formulation samples in HPLC vials with diluent to around 4 μg/mL. For example, 200 μL of mAb formulation sample and 800 μL of diluent were mixed in a HPLC. Solutions were mixed well by vortexing for up to 30 seconds for HPLC analysis. If a smaller sample volume is used, diluent volume shall be adjusted accordingly so final DTPA level remains at around 4 μg/mL. Sample solution can be used for up to 48 hours when stored at 2-8° C.

Sample Preparation for Assay of DTPA in Stock Solution and Formulation Buffer: First, dilute the DTPA in stock solution and formulation buffer to around 20 μg/mL (50 μM) with water. For example, transfer 2.0 mL of 5 mM DTPA stock solution into a 200-mL volumetric flask (50 μM, 20 μg/mL). Bring to volume with water and mix well. Second, Mix 200 μL of this diluted sample (50 μM, 20 μg/mL) and 800 μL of diluent in a HPLC vial. Mix solutions well by vortexing for up to 30 seconds for HPLC analysis. If a smaller sample volume was used, diluent volume was adjusted accordingly so final DTPA level remains at around 4 μg/mL. Sample solution could be used for up to 48 hours when stored at 2-8° C.

If any hazy solution or precipitation was observed after mixing the mAb sample with diluent, the solution was centrifuged in an Eppendorf centrifugation tube (1.5 mL) at 10,000 rpm for 5 minutes. The clear supernatant was transferred for HPLC analysis. Samples were not filtered.

Column Conditioning: When a new column was used or during the initial HPLC system set up, the column and system were conditioned by running the mobile phase for 1-2 hours and by making multiple injections of blanks, DTPA working standard solution, and a working sample solution before the start of actual sample sequence. This is important to achieve a consistent response of DTPA in its standard solutions and in mAb formulations. This HPLC method makes direct injections of samples and depends on the size-exclusion effect of narrow pore size (100 Å) and charge repulsion of a HILIC column used to elute mAb at the void and separate DTPA as a single peak for a reliable quantitation.

### System Suitability Test Procedures

Reproducibility: The relative standard deviation of the peak response for all injections of DTPA System Suitability Solution must be ≤5.0%. If the RSD is >5.0%, investigate proper peak integration, system leaks, system conditioning, or injector precision using the manufacturer's recommended tests.

Retention Time: The retention time of DTPA from a working standard solution must be between 3.5 and 4.1 min.

Tailing Factor: The tailing factor of DTPA from a working standard solution should be ≤2.0. If tailing factor is >2.0, or the retention time of DTPA is <3.5 or >4.1 min., perform one or more of the following: (a) Re-equilibrate the column; (b) Prepare fresh mobile phases; (c) Wash the column; or (d) Replace the column.

System Pressure: The column pressure was around 1900 psi after 1000 injections.

### Example 2: Calculation

The Concentration (μg/mL) of DTPA was calculated using the following formula:

$$C ( {V}_{u} / {V}_{n} ) ( {r}_{u} / {r}_{s} )$$

In which C is the concentration (μg/mL) of DTPA in the Working Standard Solution; rᵤ and rₛ are the peak responses obtained from the Working Sample and Working Standard Solution, respectively, Vᵤ is the total volume (μL) of the Working Sample preparation; Vₙ is the volume (μL) of sample used for Working Sample preparation.

The concentration of DTPA in the Working Standard Solution, C, is calculated using the weight and volume, while applying the purity value of DTPA reference material and Dilution Factor. The purity value is expressed in terms of DTPA in its salt-free form, accounting for water and residual solvent content. The concentration of Working Standard solution, C, can be calculated as:

$$[ Ws \times P ] \times D \times 1000 / Vs$$

Ws is the weight (mg) of the reference material for DTPA, P is the purity of reference material (expressed as a fraction), Vs is the volume of Stock Standard Solution (mL). D is the Dilution Factor (1/100).

### Example 3: Repeatability and Robustness of the HILIC Column for the Analysis

To test the repeatability and robustness of the methods described herein, multiple injections of various mAb-1 samples were conducted on the HILIC column used in the methods.

As shown in FIG. 2, the chromatograms show the elution profiles of a blank, a DTPA standard, and two batches of mAb-1 antibody formulations on a SIELC Newcrom BH column described herein at the wavelength of 260 nm. The antibodies are eluted in the void at about 1.0 min, Fe at about 1.8 min, while DTPA-Fe complex is eluted at about 3.7 min. Good separation of the antibodies and DTPA was observed to allow for calculation of the DTPA concentration.

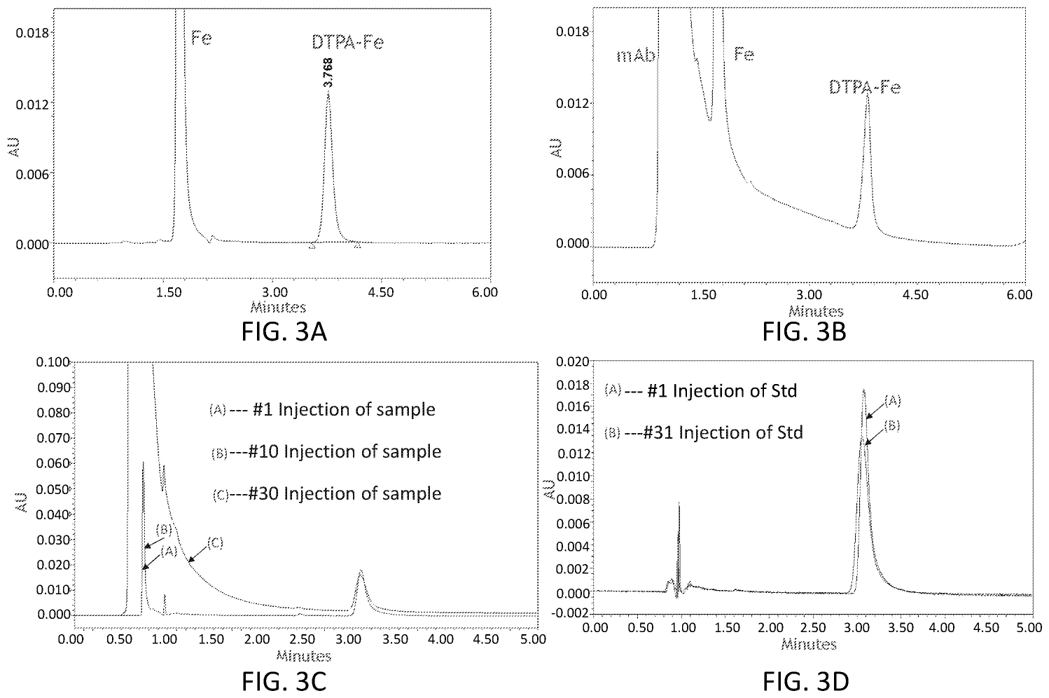

As shown in FIG. 3A, the chromatogram of the elution profile of 4 μg/mL DTPA standard solution still shows a good and clean peak of DTPA after 88 injections of the mAb-1 sample. After 88 injections, the separation of the protein and DTPA is still repeated with only slight peak tailing (FIG. 3B).

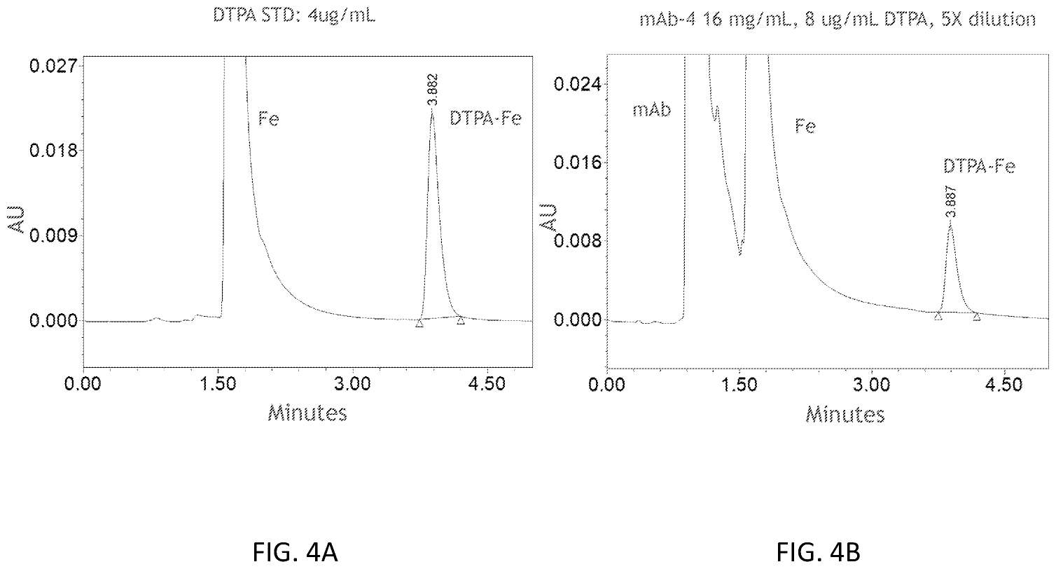

As shown in FIGS. 4A and 4B, the chromatograms showing the elution profile of a 4 μg/mL DTPA standard solution and a 5× dilution of a mAb-4 formulation solution comprising 174 mg/mL mAb-4 and 20 μg/mL DTPA, respectively. Fe is eluted at about 1.8 min. DTPA is eluted at about 3.9 min. As shown in FIG. 4B, the antibody is eluted in the void at about 1.2 min, before Fe is eluted. DTPA is eluted at about 3.9 min. Good separation of the antibodies and DTPA was observed to allow for calculation of the DTPA concentration.

To compare with the methods described herein, DTPA analysis in an mAb formulation was also performed by reverse-phase UPLC employing a narrow pore C15 column, Supelco Ascentis Express C18, 100×3.0 mm, 2 μm particular size, 90 Å. (Sigma-Aldrich, St. Louis, MO, USA) using UV detection at 260 nm (See Huang et al., J. Chrom. A, 2016, 1455:140-146). The stock DTPA standard solution contains 0.8 mg/mL of DTP in tetrabutylammonium hydroxide (TBA) buffer, pH 6.5. The sample was diluted to bring the estimated DTPA concentration down to about 4 μg/mL of DTPA in TBA buffer with 0.3 mM FeCl₃. A typical retention time for DTPA was about 2.4 minutes. The injection volume was 30 μM, column temperature was 30° C., flow rate was 0.5 mL/minute, and run time was 4 minutes.

The concentration (μg/mL) of DTPA was calculated using the same formula of Example 2: wherein the Dilution Factor is 1/200.

As shown in FIG. 3C, after 30 injections, protein peak tailing on C18 analytical column quickly deteriorates the column performance and eventually affects the DTPA quantification. As a result, analytical column needed to be replaced after about 30 injections to meet the method performance requirements. Thus, the methods described herein suit better for analyzing chelators in high concentration mAb formulations.

### Example 4: Comparison of Recovery in Diluents with Different Acid Levels

To compare the effect of the acid level on the recovery of DTPA with the methods provided herein, spike recovery of DTPA in samples comprising 120 mg/mL of a protein in the presence of different concentrations of sulfuric acid were calculated and the results are show in Table 1 below. The % recovery increased from 72.5% to 94% when the sulfuric acid concentration increased from 0.1% to 0.2%.

TABLE 1
Spiking Recovery (%)

| 0.1% Sulfuric acid | 0.15% Sulfuric acid | 0.2% Sulfuric acid |
|---|---|---|
| 72.5% | 91.7% | 94.0% |

### Example 5: Assessment of the Accuracy of the Method

To assess the accuracy of the methods provided herein, spiking recovery of DTPA in various antibody samples were conducted by diluting samples to around 4 μg/mL of DTPA first, then each was spiked with equal volume of 4 μg/mL standard solution. Formulation samples of different mAbs or protein were used for spiking recovery and the results are shown in Table 2 below. The percentage of spiking recovery of DTPA in each sample is between 91.7% and 96.5%, suggesting the method of measuring the concentration of DTPA in samples is reliable.

TABLE 2

| mAbs | Concentration (mg/mL) | Spiking Recovery % |
|---|---|---|
| mAb-7 | 27.3 | 96.5 |
| mAb-2 | 54 | 96.5 |
| mAb-3 | 49.7 | 96.5 |
| mAb-4 | 150 | 91.7 |
| mAb-4/mAb-5 1:1 | 150 | 95.0 |
| Combination | | |
| mAb-5 sample 1 | 120 | 92.5 |
| mAb-5 sample 2 | 119 | 91.7 |
| mAb-6 | 150 | 94.0 |
| Average Recovery | N/A | 94.3 |

### Example 6: Validation of Linearity of the Method

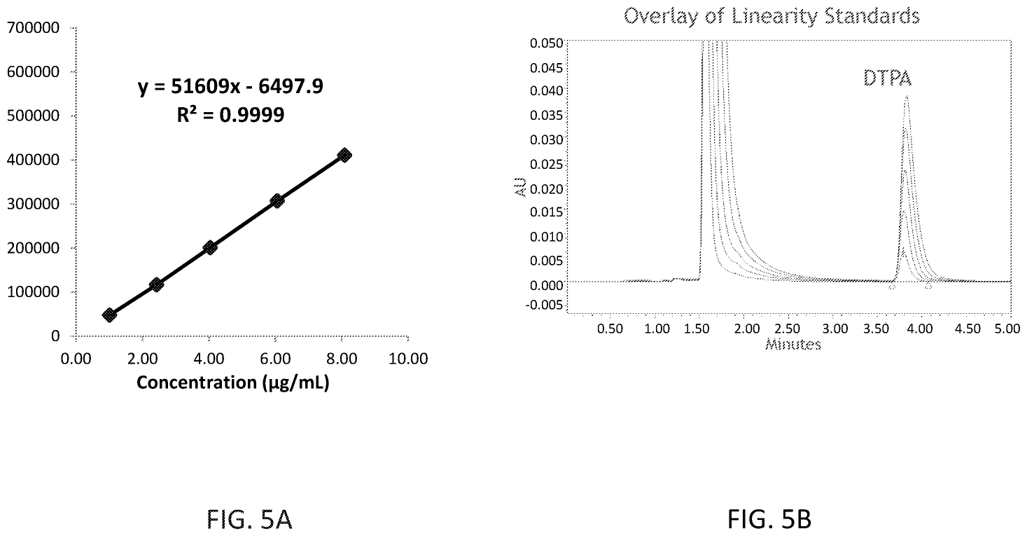

Linearity was shown using DTPA standard solutions ranging from 1 μg/mL to 8 μg/mL. The results are shown in FIG. 5A and FIG. 5B. The results suggests excellent linearity was obtained in the range of 25%-200% of Working Concertation (4 μg/mL).

### Example 7: Validation of Precision of the Method

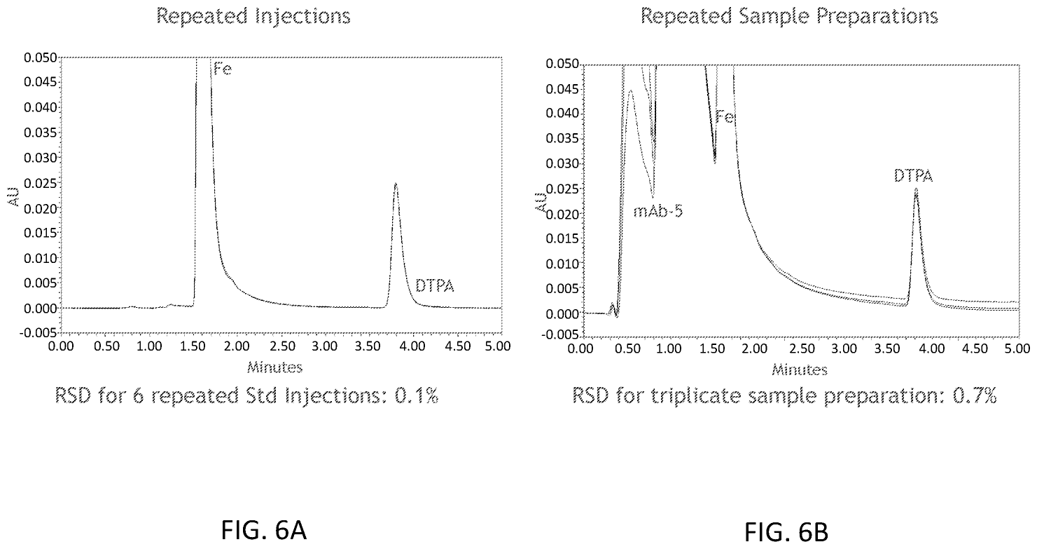

Precision of the method was tested by making six injections of DTPA standard solution at 4 μg/mL of DTPA first, and three mAb-5 sample injections. The chromatograms of the elution profiles for 6 repeated standard injections and triplicate sample preparation are shown in FIG. 6A and FIG. 6B, respectively. The relative standard deviation (RSD) for the six repeated injections was 0.1%. the RSD for the triplicate sample preparation was 0.7%.

### Example 8: Assessment of the Solution Stability

Solution stability of the working sample solution using mAb-5 was assessed for up to 3 days at 5° C. and the results are shown in Table 3. The concentrations of DTPA in the standard solution, the working sample solutions (spin down and no spin down) were stable for at least 3 days at 5° C.

TABLE 3

| | T0 | 24 hr | 48 hr | 72 hr |
|---|---|---|---|---|
| Sample Name | (μg/mL) | (μg/mL) | (μg/mL) | (μg/mL) |
| DTPA | 4.0 | 4.0 | 4.0 | 4.0 |
| Working Std | | | | |
| mAb-5 | 3.7 | 3.7 | 3.6 | 3.7 |
| Working | | | | |
| Sample | | | | |
| Solution | | | | |
| Spin down | | | | |
| mAb-5 | 3.7 | 3.7 | 3.7 | 3.7 |
| Working | | | | |
| Sample | | | | |
| Solution | | | | |
| No Spin down | | | | |

### Example 9: Case Study of mAb-6 Samples by HILIC Method

To test the stability of DTPA in a pharmaceutical formulation, DTPA concentration in various pharmaceutical formulations (PF1-5) comprising mAb-6 stored at 5° C. or 25 after 6 months was quantified using the method described herein with a SIELC Newcrom BH column and the results are shown in the Table 5 below. When stored at 5° C. or 25 after 6 months, DTPA in the formulations mAb-6 PF1, mAb-6 PF2, mAb-6 PF3, and mAb-6PF5 slightly decreased, while the concentration of mAb-6 PF4 slightly increased. The test suggests the methods described herein could be used to monitor the concentration of chelators in pharmaceutical formulations.

TABLE 5

| Formulations | T0 | 6M5C | 6M25C |
|---|---|---|---|
| mAb-6 PF1 | ND | ND | ND |
| mAb-6 PF2 | 51.8 | 51.5 | 51.0 |
| mAb-6 PF3 | 50.7 | 50.0 | 49.1 |
| mAb-6 PF4 | 50.7 | 52.5 | 51.6 |
| mAb-6 PF5 | 45.9 | 45.7 | 42.1 |

### Example 10: Chromatography Separation of EDTA and Antibody Peaks in a Sample

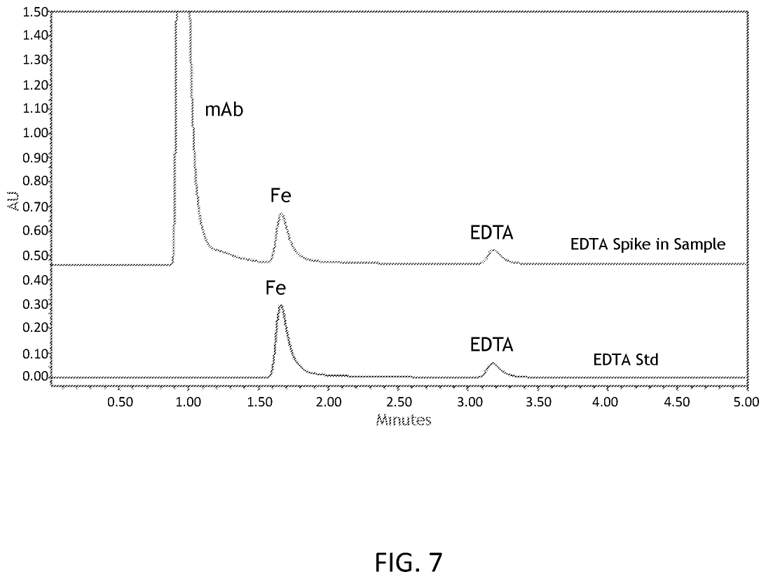

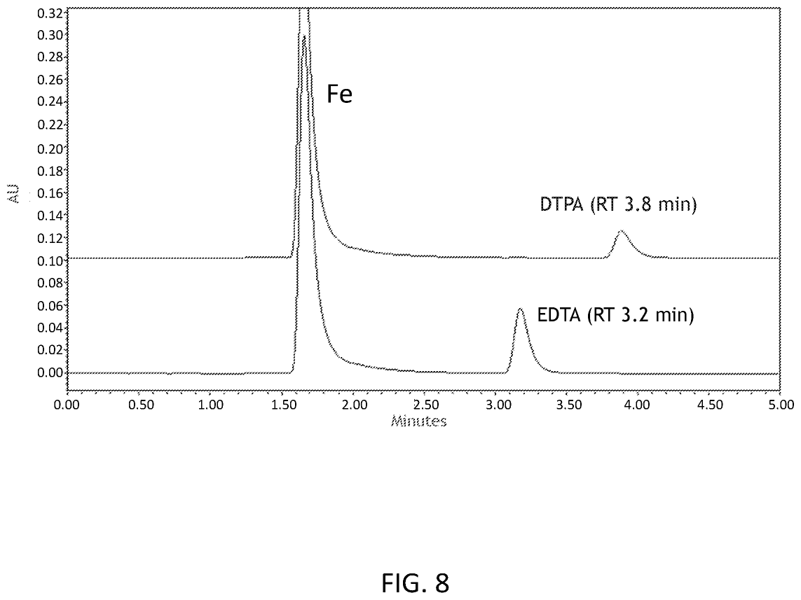

Chromatography as described in Example 1 was performed on a sample containing monoclonal antibody (mAb) and EDTA. The chromatograms of the elution profiles are shown in FIGS. 7 and 8. As shown, the chromatographic peaks for DTPA, EDTA, and monoclonal antibody were well separated from each other under HILIC conditions for DTPA as described in Example 1.

### EQUIVALENTS

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### INCORPORATION BY REFERENCE

All publications, patents, and patent applications disclosed herein are incorporated by reference to the same extent as if each individual publication, patent or patent application was specifically and individually indicated to be incorporated by reference.

What is claimed is:

1. A method of separating a chelator in a mixture comprising a protein and the chelator, the method comprising contacting the mixture with a chromatography column, wherein the chromatography column comprises a stationary phase, wherein the stationary phase:
has a charge, which is the same as the charge of the protein,
is capable of forming a non-polar interaction with the chelator, and
comprises a porous surface with a pore size that is less than the diameter of the protein.

2. The method of claim 1, which further comprises contacting the chromatography column with an elution buffer, wherein the contacting with the elution buffer occurs after the contacting of the chromatography column with the mixture.

3. The method of claim 2, wherein after contacting the chromatography column with the elution buffer, the protein is selectively eluted from the chromatography column while the chelator is retained within the chromatography column.

4. The method of any one of claims 1 to 3, further comprising eluting the chelator from the chromatography column.

5. The method of any one of claims 1 to 4, further comprising measuring the concentration of the chelator in the mixture

6. The method of claim 5, wherein measuring the concentration of the chelator in the mixture comprises contacting a standard solution with the chromatography column, wherein the standard solution comprises the chelator.

7. The method of claim 6, wherein the standard is retained within the chromatography column for at least about 3.5 min.

8. The method of claim 6 or 7, wherein the standard is retained in the column for about 3.5 min, for about 3.6 min, for about 3.7 min, for about 3.8 min, for about 3.9 min, for about 4.0 min, or for about 4.1 min.

9. The method of any one of claims 1 to 8, wherein the contacting of the mixture with the chromatography column comprises directly injecting the mixture into the chromatography column.

10. The method of any one of claims 1 to 9, wherein, prior to the contacting of the mixture with the chromatography column, the mixture is diluted in a dilution buffer.

11. The method of claim 10, wherein the mixture is diluted in the dilution buffer at about 1:1 dilution, at about 1:2 dilution, at about 1:3 dilution, at about 1:4 dilution, at about 1:5 dilution, at about 1:6 dilution, at about 1:7 dilution, at about 1:8 dilution, at about 1:9 dilution, at about 1:10 dilution, or at about 1:20 dilution, at about 1:30 dilution, at about 1:40 dilution, at about 1:50 dilution, at about 1:60 dilution, at about 1:70 dilution, at about 1:80 dilution, at about 1:90 dilution, at about 1:100 dilution, at about 1:200 dilution, at about 1:300 dilution, at about 1:400 dilution, or at about 1:500 dilution.

12. The method of any one of claims 1 to 11, which further comprises (i) obtaining a peak response from the mixture (rᵤ), (ii) obtaining a peak response from the standard solution (rₛ), (iii) or both (i) and (ii), wherein the peak response is the peak area as determined by high performance liquid chromatography (“HPLC”) or ultra performance liquid chromatography (“UPLC”) at a wavelength of 260 nm.

13. The method of claim 12, wherein the rᵤ and the rₛ are obtained by HPLC or UPLC.

14. The method of any one of claims 10 to 13, wherein the concentration (μg/mL) of the chelator is calculated as formula (I):
$$C ( {V}_{u} / {V}_{n} ) ( {r}_{u} / {r}_{s} )$$
wherein C is the concentration (μg/mL) of the chelator in the standard solution; rᵤ and rₛ are the peak responses obtained from the mixture and the standard solution, respectively, Vᵤ is the total volume (μL) of the mixture after being diluted; Vₙ is the volume (μL) of the mixture before the dilution,
wherein C is calculated as formula (II):
$$[ {W}_{s} \times P ] \times D \times 1000 / {V}_{s}$$
wherein Ws is the weight (mg) of a reference material for the chelator, P is the purity of the reference material (expressed as a fraction), Vs is the volume of the standard solution (mL), and D is the Dilution Factor (1/100).

15. A method of adjusting the concentration of a chelator in a composition comprising a protein, the method comprising measuring the concentration of the chelator present in the composition, wherein the concentration of the chelator present in the composition is measured according to the method of any one of claims 1 to 14.

16. A method of producing a composition comprising a protein, the method comprising measuring a concentration of a chelator present in the composition, wherein the concentration of the chelator present in the composition is measured according to the method of any one of claims 1 to 14.

17. The method of claim 15 or 16, which comprises increasing the concentration of the chelator if the measured concentration of the chelator is less than a reference amount (e.g., the concentration of the chelator present in a standard solution).

18. The method of claim 17, wherein the measured concentration of the chelator is less than about 5%, less than about 10%, less than about 20%, less than about 30%, less than about 40%, less than about 50%, less than about 60%, less than about 70%, less than about 80%, less than about 90%, or about 100% as compared to the reference amount.

19. The method of claim 17 or 18, wherein increasing the concentration of the chelator comprises adding an amount of the chelator to the composition.

20. The method of claim 19, which further comprises an additional measuring of the concentration of the chelator present in the composition after adding the amount of the chelator to the composition.

21. The method of claim 20, wherein the additional measuring is performed according to the method of any one of claims 1 to 14.

22. The method of any one of claims 19 to 21, wherein after adding the amount of the chelator to the composition, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference.

23. The method of claim 15 or 16, which comprises decreasing the concentration of the chelator if the measured concentration of the chelator is higher than the reference amount.

24. The method of claim 23, wherein the measured concentration of the chelator is greater than about 5%, greater than about 10%, greater than about 20%, greater than about 30%, greater than about 40%, greater than about 50%, greater than about 60%, greater than about 70%, greater than about 80%, greater than about 90%, greater than about 100%, greater than about 125%, greater than about 150%, greater than about 175%, or greater than about 200% as compared to the reference amount.

25. The method of claim 23 or 24, wherein decreasing the concentration of the chelator comprises adding a diluent to the composition, increasing the concentration of the protein in the composition, or both.

26. The method of claim 25, which further comprises an additional measuring of the concentration of the chelator present in the composition after the decreasing.

27. The method of claim 26, wherein the additional measuring is performed according to the method of any one of claims 1 to 14.

28. The method of any one of claims 23 to 27, wherein after the decreasing, the concentration of the chelator present in the composition is at least about 90%, at least about 91%, at least about 92%, at least about 93%, at least about 94%, at least about 95%, at least about 96%, at least about 97%, at least about 98%, at least about 99%, or about 100% as compared to the reference.

29. The method of any one of claims 1 to 28, wherein the chromatography column and the protein have positive charge.

30. The method of any one of claims 1 to 29, wherein the chromatography column is capable of eluting a protein, wherein the diameter of the protein is less than about 70 Å, less than about 80 Å, less than about 90 Å, less than about 100 Å, less than about 110 Å, less than about 120 Å, less than about 130 Å, less than about 140 Å, less than about 150 Å, less than about 160 Å, less than about 170 Å, or less than about 180 Å.

31. The method of any one of claims 1 to 30, wherein the chromatography column is capable of eluting a protein, wherein the diameter of the protein is between about 70 Å and about 180 Å, about between 70 Å and about 170 Å, between about 70 Å and about 160 Å, between about 70 Å and about 150 Å, between 70 about Å and about 140 Å, between about 70 Å and about 130 Å, between about 70 Å and about 120 Å, between about 70 Å and about 110 Å, between about 70 Å and about 100 Å, between about 80 Å and about 180 Å, between about 80 Å and about 170 Å, between about 80 Å and about 160 Å, between about 80 Å and about 150 Å, between about 80 Å and about 140 Å, between about 80 Å and about 130 Å, between about 80 Å and about 120 Å, between about 80 Å and about 110 Å, between about 80 Å and about 100 Å, between about 90 Å and about 180 Å, between about 90 Å and about 170 Å, between about 90 Å and about 160 Å, between about 90 Å and about 150 Å, between about 90 Å and about 140 Å, between about 90 Å and about 130 Å, between about 90 Å and about 120 Å, between about 90 Å and about 110 Å, between about 90 Å and about 100 Å, between about 100 Å and about 180 Å, between about 100 Å and about 170 Å, between about 100 Å and about 160 Å, between about 100 Å and about 150 Å, between about 100 Å and about 140 Å, between about 100 Å and about 130 Å, between about 100 Å and about 120 Å, between about 100 Å and about 110 Å, between about 110 Å and about 180 Å, between about 110 Å and about 170 Å, between about 110 Å and about 160 Å, between about 110 Å and about 150 Å, between about 110 Å and about 140 Å, between about 110 Å and about 130 Å, between about 110 Å and about 120 Å, between about 120 Å and about 180 Å, between about 120 Å and about 170 Å, between about 120 Å and about 160 Å, between about 120 Å and about 150 Å, between about 120 Å and about 140 Å, between about 120 Å and about 130 Å, between about 130 Å and about 180 Å, between about 130 Å and about 170 Å, between about 130 Å and about 160 Å, between about 130 Å and about 150 Å, between about 130 Å and about 140 Å, between about 140 Å and about 180 Å, between about 140 Å and about 170 Å, between about 140 Å and about 160 Å, between about 140 Å and about 150 Å, between about 150 Å and about 180 Å, between about 150 Å and about 170 Å, between about 150 Å and about 160 Å, between about 160 Å and about 180 Å, between about 160 Å and about 170 Å, or between about 170 Å and about 180 Å.

32. The method of any one of claims 1 to 31, wherein the pore size is no more than about 110 Å.

33. The method of any one of claims 1 to 32, wherein the pore size is about 110 Å, about 100 Å, about 90 Å, about 80 Å, or about 70 Å.

34. The method of any one of claims 1 to 33, wherein the chelator comprises DTPA, EDTA, or both.

35. The method of any one of claims 1 to 34, wherein the chromatography column is an hydrophilic interaction chromatography (HILIC) column.

36. The method of any one of claims 1 to 35, wherein the chromatography column is packed with a stationary phase comprising multimode hydrophobic ligand and a positive charged terminal functional group.

37. The method of claim 36, wherein the positive charged terminal functional group comprises an amino group.

38. The method of any one of claims 1 to 37, wherein the column is Newcrom BH column.

39. The method of any one of claims 1 to 38, wherein the chromatography is performed using a mobile phase comprising a buffer.

40. The method of claim 39, wherein the buffer comprises acetonitrile, sulfuric acid, FeCl₃, or a combination thereof.

41. The method of claim 40, wherein the acetonitrile is present in the buffer at a concentration of about 0.5%, about 1%, about 1.5%, about 2%, about 2.5%, or about 3%.

42. The method of claim 40 or 41, wherein the sulfuric acid is present in the buffer at a concentration of about 0.05%, about 0.1%, about 0.2%, about 0.3%, about 0.4%, about 0.5%, about 0.6%, about 0.7%, about 0.8%, about 0.9%, or about 1%.

43. The method of claim 42, wherein the sulfuric acid is present in the buffer at a concentration of about 0.15% to 0.20%.

44. The method of any one of claims 40 to 43, wherein the FeCl₃ is present in the buffer at a concentration of about 0.1 mM, about 0.2 mM, about 0.3 mM, about 0.4 mM, about 0.5 mM, about 0.6 mM, about 0.7 mM, about 0.8 mM, about 0.9 mM, or about 1.0 mM.

45. The method of any one of claims 40 to 44, wherein the buffer comprises 2% Acetonitrile, 0.2% sulfuric acid, and 0.6 mM FeCl₃.

46. The method of any one of claims 43 to 45, wherein the buffer comprises 2% Acetonitrile, 0.15% sulfuric acid, 0.6 mM FeCl₃.

47. The method of any one of claims 39 to 46, wherein the buffer has a pH from about 1.0 to about 5.0, or 1.0 to about 4.5, or about 1.0 to about 4.0.

48. The method of any one of claims 39 to 47, wherein the buffer has a pH of about 0.5, about 1.0, about 1.5, about 2.0, about 2.5, about 3.0, about 3.5, about 4.0, about 4.5, or about 5.0.

49. The method of any one of claims 39 to 48, wherein the buffer has a pH of about 1.0 to about 4.0, or any pH in between.

50. The method of claim 10 or 11, wherein the dilution buffer is the buffer of any one of claims 39-49.

51. The method of any one of claims 1 to 50, wherein the concentration of the protein in the mixture is about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, or about 300 mg/mL.

52. The method of any one of claims 1 to 51, wherein the protein is conjugated to a drug, such that the mixture or the composition comprises a protein-drug conjugate (PDC).

53. The method of claim 52, wherein the protein-drug conjugate comprises an antibody-drug conjugate (ADC).

54. The method of any one of claims 1 to 53, wherein the protein comprises an antibody or antigen binding portion thereof.

55. The method of any one of claims 1 to 51, wherein the protein comprises a fusion protein.

56. The method of claim 55, wherein the fusion protein comprises a half-life extending moiety.

57. The method of claim 56, wherein the half-life extending moiety comprises an Fc.

58. The method of claim 57, wherein the fusion protein comprises an Fc fused to an antibody or antigen binding portion thereof.

59. A composition produced according to the method of any one of claims 16 to 58.

60. The composition of claim 59, which further comprises a pharmaceutically acceptable excipient.
